(19) United States
(12) Patent Application Publication
(10) Pub. No.: US 2020/0291370 A1
(43) Pub. Date: Sep. 17, 2020

(54) Mutant Cas Proteins

(71) Applicant: President and Fellows of Harvard College, Cambridge, MA (US)

(72) Inventor: Alejandro Chavez, New York, NY (US)

(73) Assignee: President and Fellows of Harvard College, Cambridge, MA (US)

(21) Appl. No.: 16/086,067

(22) PCT Filed: Mar. 16, 2017
(86) PCT No.: PCT/US2017/022626

§ 371 Date: Sep. 18, 2018

Related U.S. Application Data
(60) Provisional application No. 62/310,018, filed on Mar. 18, 2016.

(51) Int. Cl. C12N 9/22

(52) U.S. Cl. CPC C12N 9/22

(57) ABSTRACT

CRISPR/Cas Systems are provided where mutant Cas9 proteins or Cas9 proteins are provided that have improved binding to a target nucleic acid sequence having a functional PAM compared to wild type Cas9 or that bind to a target nucleic acid that lacks a functional PAM.

### RELATED APPLICATION DATA

This application claims priority to U.S. Provisional Application No. 62/310,018 filed on Mar. 18, 2016 which is hereby incorporated herein by reference in entirety for all purposes.

### STATEMENT OF GOVERNMENT INTERESTS

This invention was made with government support under 5P50HG005550-05, 1RM1HG008525-01, and 5T32CA009216-34 awarded by National Institutes of Health. The government has certain rights in the invention.

### BACKGROUND

The CRISPR type II system is a recent development that has been efficiently utilized in a broad spectrum of species. See Friedland, A. E., et al., Heritable genome editing in C. elegans via a CRISPR-Cas9 system. Nat Methods, 2013. 10(8): p. 741-3, Mali, P., et al., RNA-guided human genome engineering via Cas9. Science, 2013. 339(6121): p. 823-6, Hwang, W. Y., et al., Efficient genome editing in zebrafish using a CRISPR-Cas system. Nat Biotechnol, 2013, Jiang, W., et al., RNA-guided editing of bacterial genomes using CRISPR-Cas systems. Nat Biotechnol, 2013, Jinek, M., et al., RNA-programmed genome editing in human cells. elife, 2013. 2: p. e00471, Cong, L., et al., Multiplex genome engineering using CRISPR/Cas systems. Science, 2013. 339(6121): p. 819-23, Yin, H., et al., Genome editing with Cas9 in adult mice corrects a disease mutation and phenotype. Nat Biotechnol, 2014. 32(6): p. 551-3. CRISPR is particularly customizable because the active form consists of an invariant Cas9 protein and an easily programmable guide RNA (gRNA). See Jinek, M., et al., A programmable dual-RNA-guided DNA endonuclease in adaptive bacterial immunity. Science, 2012. 337(6096): p. 816-21. Of the various CRISPR orthologs, the Streptococcus pyogenes (Sp) CRISPR is the well-characterized and widely used. The Cas9-gRNA complex first probes DNA for the protospacer-adjacent motif (PAM) sequence (-NGG for Sp Cas9), after which Watson-Crick base-pairing between the gRNA and target DNA proceeds in a ratchet mechanism to form an R-loop. Following formation of a ternary complex of Cas9, gRNA, and target DNA, the Cas9 protein generates two nicks in the target DNA, creating a blunt double-strand break (DSB) that is predominantly repaired by the non-homologous end joining (NHEJ) pathway or, to a lesser extent, template-directed homologous recombination (HR). CRISPR methods are disclosed in U.S. Pat. Nos. 9,023,649 and 8,697,359.

### SUMMARY

The disclosure provides mutant Cas proteins, such as mutant Cas9 proteins. The disclosure provides methods of altering a target nucleic acid sequence in a cell including providing to the cell a mutant Cas9 protein or a Cas9 protein having an accessory DNA binding domain attached thereto and providing to the cell a guide RNA including a spacer sequence and a tracr mate sequence forming a crRNA and a tracr sequence wherein the guide RNA and the Cas9 protein (whether mutant or including a DNA binding domain) form a co-localization complex with the target nucleic acid. The tracr sequence and the crRNA sequence may be separate or connected by the linker. According to one aspect, the crRNA and the tracr sequence of the guide RNA are separate sequences. According to one aspect, the mutant Cas9 protein or a Cas9 protein having an accessory DNA binding domain attached thereto is provided to the cell by introducing into the cell a first foreign nucleic acid encoding the mutant Cas9 protein the Cas9 protein having an accessory DNA binding domain attached thereto and wherein the guide RNA is provided to the cell by introducing into the cell a second foreign nucleic acid encoding the guide RNA, wherein the guide RNA and the mutant Cas9 protein or the Cas9 protein having an accessory DNA binding domain attached thereto are expressed, and wherein the guide RNA and the mutant Cas9 protein or the Cas9 protein having an accessory DNA binding domain attached thereto co-localize to the target nucleic acid. According to one aspect, the mutant Cas9 protein or the Cas9 protein having an accessory DNA binding domain attached thereto is an enzymatically active Cas9 protein that cleaves both strands of the target nucleic acid. According to one aspect, the mutant Cas9 protein or the Cas9 protein having an accessory DNA binding domain attached thereto is a nickase which nicks or cuts or cleaves a single strand of the target nucleic acid sequence. According to one aspect, the mutant Cas9 protein or the Cas9 protein having an accessory DNA binding domain attached thereto is a nuclease null or nuclease deficient Cas9 protein to which may be attached a transcriptional regulator, such as a transcriptional activator or transcriptional repressor and the target nucleic acid is modulated such as being upregulated or downregulated. According to one aspect, the cell is in vitro, in vivo or ex vivo. According to one aspect, the cell is a eukaryotic cell or prokaryotic cell. According to one aspect, the cell is a bacteria cell, a yeast cell, a fungal cell, a mammalian cell, a plant cell or an animal cell. According to one aspect, the selected RNA sequence is between about 10 and about 10,000 nucleotides. According to one aspect, the target nucleic acid is genomic DNA, mitochondrial DNA, plastid DNA, viral DNA, exogenous DNA or cellular RNA.

The disclosure provides a mutant Cas9 protein having target nucleic acid binding activity in the absence of an adjacent functional protospacer adjacent motif. The disclosure provides that the mutant Cas9 protein include one or more amino acid mutations, such as a negatively charged amino acid to a neutral charged amino acid, a negatively charged amino acid to a positively charged amino acid or a neutral charged amino acid to a positively charged amino acid. The disclosure provides that the mutant Cas9 protein include one or more amino acid mutations that result in the mutant Cas9 protein having a lower electrostatic repulsion to DNA compared to wild type or unmutated Cas protein. The disclosure provides that the mutant Cas9 protein include one or more mutations, such as G1104K, L1111H, D1135Y and N1317K. The disclosure provides that the mutant Cas9 protein have nuclease activity. The disclosure provides that the mutant Cas9 protein is a nickase. The disclosure provides that the mutant Cas9 protein is nuclease null. The disclosure provides that the mutant Cas9 protein have a transcriptional regulator attached thereto.

The disclosure provides a mutant Cas9 protein having increased target nucleic acid binding activity in the presence of an adjacent functional protospacer adjacent motif. The disclosure provides that the mutant Cas9 protein include one or more amino acid mutations, such as a negatively charged amino acid to a neutral charged amino acid, a negatively charged amino acid to a positively charged amino acid or a neutral charged amino acid to a positively charged amino acid. The disclosure provides that the mutant Cas9 protein include one or more amino acid mutations that result in the mutant Cas9 protein having a lower electrostatic repulsion to DNA compared to wild type or unmutated Cas protein.

The disclosure provides a mutant Cas9 protein bound to a target nucleic acid, wherein the target nucleic acid lacks an adjacent functional protospacer adjacent motif.

The disclosure provides a mutant Cas9 protein including one or more mutations, such as G1104K, L1111H, D1135Y andN1317K.

The disclosure provides a method of making a mutant Cas9 protein including expressing a nucleic acid sequence encoding a Cas9 protein including one or more mutations, such as a negatively charged amino acid to a neutral charged amino acid, a negatively charged amino acid to a positively charged amino acid or a neutral charged amino acid to a positively charged amino acid. The disclosure provides that the one or more mutations, such as G1104K, L1111H, D1135Y and N1317K.

The disclosure provides a method of altering a target nucleic acid in a cell including providing to the cell a mutant Cas9 protein including one or more mutations, such as a negatively charged amino acid to a neutral charged amino acid, a negatively charged amino acid to a positively charged amino acid or a neutral charged amino acid to a positively charged amino acid, providing to the cell a guide RNA including a spacer sequence complementary to a target nucleic acid, wherein the guide RNA and the mutant Cas9 protein form a co-localization complex with the target nucleic acid, and the target nucleic acid is altered. The disclosure provides that the one or more mutations may be G1104K, L1111H, D1135Y or N1317K. The disclosure provides that mutant Cas9 protein is an enzymatically active Cas9 and the target nucleic acid is cleaved by the mutant Cas9 protein. The disclosure provides that the mutant Cas9 protein is a nickase and one strand of the target nucleic acid is cleaved by the mutant Cas9 protein. The disclosure provides that the mutant Cas9 protein is a nuclease null Cas9 and wherein a transcriptional regulator is attached to either the mutant Cas9 protein or the guide RNA and the target nucleic acid is regulated. The disclosure provides that the mutant Cas9 protein is provided to the cell by introducing into the cell a first foreign nucleic acid encoding the mutant Cas9 protein and wherein the guide RNA is provided to the cell by introducing into the cell a second foreign nucleic acid encoding the guide RNA, and wherein the guide RNA and the mutant Cas9 protein are expressed. The disclosure provides that the cell is in vitro, in vivo or ex vivo. The disclosure provides that the cell is a eukaryotic cell or prokaryotic cell. The disclosure provides that the cell is a bacteria cell, a yeast cell, a fungal cell, a mammalian cell, a plant cell or an animal cell. The disclosure provides that the target nucleic acid is genomic DNA, mitochondrial DNA, plastid DNA, viral DNA, or exogenous DNA.

The disclosure provides a cell including a mutant Cas9 protein including one or more mutations, such as a negatively charged amino acid to a neutral charged amino acid, a negatively charged amino acid to a positively charged amino acid or a neutral charged amino acid to a positively charged amino acid, and a guide RNA and wherein the guide RNA and the Cas9 protein are members of a co-localization complex for the target nucleic acid. The disclosure provides that the one or more mutations may be G1104K, L1111H, D1135Y and N1317K. The disclosure provides that the cell is a eukaryotic cell or prokaryotic cell. The disclosure provides that the cell is a bacteria cell, a yeast cell, a fungal cell, a mammalian cell, a plant cell or an animal cell.

The disclosure provides a cell including a first foreign nucleic acid encoding a mutant Cas9 protein including one or more mutations, such as a negatively charged amino acid to a neutral charged amino acid, a negatively charged amino acid to a positively charged amino acid and a neutral charged amino acid to a positively charged amino acid, and a second foreign nucleic acid encoding a guide RNA and wherein the guide RNA and the mutant Cas9 protein are members of a co-localization complex for a target nucleic acid. The disclosure provides that the one or more mutations may be G1104K, L1111H, D1135Y and N1317K. The disclosure provides that the cell is a eukaryotic cell or prokaryotic cell. The disclosure provides that the cell is a bacteria cell, a yeast cell, a fungal cell, a mammalian cell, a plant cell or an animal cell.

The disclosure provides an RNA guided nucleic acid binding protein having one or more accessory DNA binding domains attached thereto.

The disclosure provides an RNA guided nucleic acid binding protein having one or more accessory DNA binding domains attached thereto and having target nucleic acid binding activity in the absence of an adjacent functional protospacer adjacent motif. The disclosure provides that the RNA guided nucleic acid binding protein has nuclease activity. The disclosure provides that the RNA guided nucleic acid binding protein is a nickase. The disclosure provides that the RNA guided nucleic acid binding protein is a nuclease null Cas9 protein. The disclosure provides that the RNA guided nucleic acid binding protein has a transcriptional regulator attached thereto.

The disclosure provides an RNA guided nucleic acid binding protein having one or more accessory DNA binding domains attached thereto and having increased target nucleic acid binding activity in the presence of an adjacent functional protospacer adjacent motif, compared to wild type RNA guided nucleic acid binding protein.

The disclosure provides an RNA guided nucleic acid binding protein having one or more accessory DNA binding domains attached thereto bound to a target nucleic acid, wherein the target nucleic acid lacks an adjacent functional protospacer adjacent motif.

The disclosure provides a method of improving binding of an RNA guided nucleic acid binding protein to a first target nucleic acid including combining an RNA guided nucleic acid binding protein having an accessory DNA binding domain attached thereto, a guide RNA having a spacer sequence complementary to the first target nucleic acid sequence and the first target nucleic acid under conditions where the RNA guided nucleic acid binding protein binds to the first target nucleic acid and the accessory DNA binding domain binds to an accessory target nucleic acid.

The disclosure provides a method of altering expression of a target nucleic acid in a cell including providing to the cell a Cas9 protein having an accessory DNA binding domain attached thereto, providing to the cell a guide RNA including a spacer sequence complementary to a target nucleic acid, wherein the guide RNA and the Cas9 protein form a co-localization complex with the target nucleic acid, and the target nucleic acid is altered. The disclosure provides that the Cas9 protein is an enzymatically active Cas9 and the target nucleic acid is cleaved by the Cas9 protein. The disclosure provides that the Cas9 protein is a nickase and one strand of the target nucleic acid is cleaved by the Cas9 protein. The disclosure provides that the mutant Cas9 protein is a nuclease null Cas9 and wherein a transcriptional regulator is attached to either the Cas9 protein or the guide RNA and the target nucleic acid is regulated. The disclosure provides that the Cas9 protein is provided to the cell by introducing into the cell a first foreign nucleic acid encoding the Cas9 protein and wherein the guide RNA is provided to the cell by introducing into the cell a second foreign nucleic acid encoding the guide RNA, and wherein the guide RNA and the Cas9 protein are expressed. The disclosure provides that the cell is in vitro, in vivo or ex vivo. The disclosure provides that the cell is a eukaryotic cell or prokaryotic cell. The disclosure provides that the cell is a bacteria cell, a yeast cell, a fungal cell, a mammalian cell, a plant cell or an animal cell. The disclosure provides that the target nucleic acid is genomic DNA, mitochondrial DNA, plastid DNA, viral DNA, or exogenous DNA.

The disclosure provides a cell including a Cas9 protein having an accessory DNA binding domain attached thereto, and a guide RNA and wherein the guide RNA and the Cas9 protein are members of a co-localization complex for the target nucleic acid. The disclosure provides that the cell is a eukaryotic cell or prokaryotic cell. The disclosure provides that the cell is a bacteria cell, a yeast cell, a fungal cell, a mammalian cell, a plant cell or an animal cell.

The disclosure provides a cell including a first foreign nucleic acid encoding a Cas9 protein having an accessory DNA binding domain attached thereto, and a second foreign nucleic acid encoding a guide RNA and wherein the guide RNA and the Cas9 protein are members of a co-localization complex for a target nucleic acid. The disclosure provides that the cell is a eukaryotic cell or prokaryotic cell. The disclosure provides that the cell is a bacteria cell, a yeast cell, a fungal cell, a mammalian cell, a plant cell or an animal cell.

Further features and advantages of certain embodiments of the present invention will become more fully apparent in the following description of embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present embodiments will be more fully understood from the following detailed description of illustrative embodiments taken in conjunction with the accompanying drawings in which:

FIG. 1A and FIG. 1B are graphs of data generated by a fluorescent reporter assay measuring the ability of various mutant Cas9 proteins to activate gene expression. To generate the data depicted in FIG. 1A, nuclease positive Cas9 or point mutated versions of Cas9 were directed to a fluorescent reporter assay containing either an NGG PAM upstream of an iRFP713 fluorescent reporter or a mixture of three plasmids containing either an NGA, NAG, or NGC PAM upstream of a tdtomato reporter. To generate the data depicted in FIG. 1B, nuclease null Cas9 and nuclease null Cas9 fused to a variety of DNA interacting domains were directed to the same fluorescent reporters as in panel FIG. 1A. Activation for all Cas9 proteins was achieved by utilizing a guide RNA containing MS2 hairpins which enabled the recruitment of the MS2-binding protein-p65-hsf1 activator. For FIG. 1A experiments involving nuclease competent Cas9, a 14nt gRNA was used. For FIG. 1B experiments involving nuclease null Cas9 fusions, a 20nt gRNA was used. For the bar graph data for the various Cas9 proteins, activity on NAG/NGC/NGA is represented by the right bar in blue and activity on NGG is represented by the left bar in orange.

FIG. 2A and FIG. 2B are graphs of data of relative RNA expression as a measure of the ability of various mutant Cas9 proteins to activate endogenous gene expression. In FIG. 2A, nuclease positive Cas9 or point mutated versions were simultaneously directed to the promoter of MIAT and TTN through interaction with an NGG PAM at the target locus. In FIG. 2B, nuclease null Cas9 and nuclease null Cas9 fused to a variety of DNA interacting domains were simultaneously directed to the promoter of MIAT and TTN through interaction with an NGG PAM at the target locus. Activation for all Cas9 proteins was achieved by utilizing a guide RNA containing MS2 hairpins which enabled the recruitment of the MS2-binding protein-p65-hsf1 activator. For FIG. 2A experiments involving nuclease competent Cas9, a 14nt gRNA was used. For FIG. 2B experiments involving nuclease null Cas9 fusions, a 20nt gRNA was employed. Relative RNA expression for each experiment is normalized to cells transfected with only gRNA but no Cas9 component. For the bar graph data for the various Cas9 proteins, MIAT is represented by the right bar in blue and TTN is represented by the left bar in orange.

FIG. 3A and FIG. 3B are graphs of data demonstrating Cas9 DNA binding domain fusions enhance DNA binding activity leading to improved repression and target locus modification. In FIG. 3A, various nuclease null versions of Cas9 were directed to a constitutively active fluorescent reporter assay. In the presence of Cas9, reporter activation is inhibited and a decrease in fluorescence is observed and quantified through flow cytometry. In FIG. 3B, various nuclease competent versions of Cas9 were directed to a reporter construct designed to test the efficiency of DNA modification through the generation of a small deletion upon Cas9 cutting of the target construct. For all experiments n=2 independent biological replicates and error bars represent ±1 standard deviation.

FIG. 4A and FIG. 4B are graphs of data demonstrating DNA binding domain fusions to Cas9 orthologues enhance target site activation or target site modification. In FIG. 4A, MS2-p65-hsf1 activator along with various nuclease null versions of Sa-Cas9 in conjunction with a gRNA with MS2-haripins were directed to a fluorescent reporter assay. In the presence of Cas9 binding the transcription of a downstream fluorescent reporter is induced and fluorescence is quantified through flow cytometry. n=2 independent biological replicates and error bars represent ±1 standard deviation. In FIG. 4B, various nuclease competent versions of ST1-Cas9 were directed to a reporter construct designed to test the efficiency of DNA modification through the generation of a small deletion upon Cas9 cutting of the target construct.

FIG. 5 is a graph of data demonstrating improved Cas9 cutting at endogenous loci by fusing additional DNA binding sequences to Cas9. Cells were transfected with a mixture of gRNAs targeted the noted loci along with the corresponding Cas9 variant. The percentage of modified alleles are quantified (% indel) and plotted across each locus. n=2 independent biological replicates, error bars are ±s.e.m. For bar graph data for each target locus, Cas9 is represented by the blue bar to the right, Cas9-ctb is represented by the center bar in orange and Cas9-sso7d is represented by the right bar in grey.

### DETAILED DESCRIPTION

The present disclosure provides mutant RNA guided nucleic acid binding proteins, such as Cas proteins, or RNA guided nucleic acid binding proteins, such as Cas proteins, including one or more foreign DNA binding domains for use in an RNA guided DNA binding system, such as a CRISPR/Cas system which utilizes a guide RNA which includes a spacer sequence, a tracr mate sequence and a tracr sequence. Exemplary Cas proteins include orthologs thereof. Exemplary Cas proteins include Cas9 proteins. Exemplary Cas proteins include Cpf1. It is to be understood that where the disclosure specifically mentions Cas9 proteins, other Cas proteins or RNA guided nucleic acid binding proteins may be used.

According to one aspect, the mutant Cas protein, the Cas including one or more foreign DNA binding domains or the guide RNA may have one or more transcriptional regulator proteins or domains attached, bound, connected or fused thereto. According to one aspect, the transcriptional regulator protein or domain is a transcriptional activator. According to one aspect, the transcriptional regulator protein or domain upregulates expression of the target nucleic acid. According to one aspect, the transcriptional regulator protein or domain is a transcriptional repressor. According to one aspect, the transcriptional regulator protein or domain downregulates expression of the target nucleic acid. Transcriptional activators and transcriptional repressors can be readily identified by one of skill in the art based on the present disclosure.

According to one aspect, the mutant Cas protein, the Cas including one or more foreign DNA binding domains, or the guide RNA may have one or more detectable proteins or domains or labels or markers attached, bound, connected or fused thereto, which can then be detected or imaged to identify the location of the target nucleic acid sequence. Detectable labels or markers can be readily identified by one of skill in the art based on the present disclosure.

According to certain aspects of the present disclosure, the guide RNA is capable of binding to a target nucleic acid and otherwise complexing with an RNA guided binding protein of a CRISPR/Cas system. The RNA guided binding protein may be an RNA guided DNA binding protein or it may be an RNA guided RNA binding protein. According to this aspect, the spacer sequence is designed to bind to a target DNA sequence or a target RNA sequence so as to form a colocalization complex of the guide RNA and the RNA guided binding protein and either the target DNA sequence or target RNA sequence.

According to certain aspects of the present disclosure, the mutant Cas protein, the Cas including one or more foreign DNA binding domains, or guide RNA include one or more functional groups attached, connected, bound or fused thereto at locations which do not significantly interact with or otherwise prevent the colocalization of the guide RNA and the mutant Cas protein or the Cas including one or more foreign DNA binding domains with the target nucleic acid, which may be DNA or RNA.

According to certain aspects, the guide RNA and mutant Cas protein or the Cas protein with the foreign DNA binding protein attached thereto are foreign to the cell into which they are introduced. According to this aspect, the guide RNA, and mutant Cas protein or the Cas protein with the foreign DNA binding protein attached thereto are nonnaturally occurring in the cell in which they are presented. To this extent, cells may be genetically engineered or genetically modified to include the CRISPR systems described herein.

The present disclosure provides methods of targeting nucleic acids for alteration, editing or transcriptional regulation using a mutant Cas protein described herein or using an RNA guided nucleic acid binding protein which includes a foreign DNA binding domain. According to one aspect, one or more vectors are used to introduce one or more nucleic acids encoding a CRISPR system, i.e. a mutant Cas9 protein or a Cas9 protein having an accessory DNA binding domain and a guide RNA, and optionally a donor nucleic acid sequence, into a cell such as a eukaryotic cell, for alteration, editing or transcriptional regulation. The nucleic acids are expressed and the CRISPR system cuts or nicks the target nucleic acid or otherwise delivers a transcriptional regulator to the target nucleic acid. Together, a guide RNA and a mutant Cas9 protein or a Cas9 protein having an accessory DNA binding domain are referred to as a co-localization complex as that term is understood by one of skill in the art to the extent that the guide RNA and the mutant Cas9 protein or a Cas9 protein having an accessory DNA binding domain complex with a target nucleic acid. According to certain aspects, a vector may include one or more nucleic acids encoding a mutant Cas9 protein or a Cas9 protein having an accessory DNA binding domain, a guide RNA and/or a donor nucleic acid sequence. According to certain aspects, one or more nucleic acids encoding a mutant Cas9 protein or a Cas9 protein having an accessory DNA binding domain, a guide RNA and/or a donor nucleic acid sequence may be present within the same vector or present within different vectors. According to one aspect, a vector is utilized to deliver the one or more nucleic acids encoding a mutant Cas9 protein or a Cas9 protein having an accessory DNA binding domain, a guide RNA and/or a donor nucleic acid sequence into the cell for expression by the cell.

Exemplary Cas9 for Mutation or for Attaching an Accessory DNA Binding Domain

RNA guided DNA binding proteins are readily known to those of skill in the art to bind to DNA for various purposes. Such DNA binding proteins may be naturally occurring. DNA binding proteins having nuclease activity are known to those of skill in the art, and include naturally occurring DNA binding proteins having nuclease activity, such as Cas9 proteins present, for example, in Type II CRISPR systems. Such Cas9 proteins and Type II CRISPR systems are well documented in the art. See Makarova et al., Nature Reviews, Microbiology, Vol. 9, June 2011, pp. 467-477 including all supplementary information hereby incorporated by reference in its entirety.

In general, bacterial and archaeal CRISPR-Cas systems rely on short guide RNAs in complex with Cas proteins to direct degradation of complementary sequences present within invading foreign nucleic acid. See Deltcheva, E. et al. CRISPR RNA maturation by trans-encoded small RNA and host factor RNase III. Nature 471, 602-607 (2011); Gasiunas, G., Barrangou, R., Horvath, P. & Siksnys, V. Cas9-crRNA ribonucleoprotein complex mediates specific DNA cleavage for adaptive immunity in bacteria. Proceedings of the National Academy of Sciences of the United States of America 109, E2579-2586 (2012); Jinek, M. et al. A programmable dual-RNA-guided DNA endonuclease in adaptive bacterial immunity. Science 337, 816-821 (2012); Sapranauskas, R. et al. The Streptococcus thermophilus CRISPR/Cas system provides immunity in Escherichia coli. Nucleic acids research 39, 9275-9282 (2011); and Bhaya, D., Davison, M. & Barrangou, R. CRISPR-Cas systems in bacteria and archaea: versatile small RNAs for adaptive defense and regulation. Annual review of genetics 45, 273-297 (2011). A recent in vitro reconstitution of the S. pyogenes type II CRISPR system demonstrated that crRNA (“CRISPR RNA”) fused to a normally trans-encoded tracrRNA (“trans-activating CRISPR RNA”) is sufficient to direct Cas9 protein to sequence-specifically cleave target DNA sequences matching the crRNA. Expressing a gRNA homologous to a target site results in Cas9 recruitment and degradation of the target DNA. See H. Deveau et al., Phage response to CRISPR-encoded resistance in Streptococcus thermophilus. Journal of Bacteriology 190, 1390 (February 2008). Additional useful Cas proteins are from S. thermophilic or S. aureus.

Three classes of CRISPR systems are generally known and are referred to as Type I, Type II or Type III). According to one aspect, a particular useful enzyme according to the present disclosure to cleave dsDNA is the single effector enzyme, Cas9, common to Type II. See K. S. Makarova et al., Evolution and classification of the CRISPR-Cas systems. Nature reviews. Microbiology 9, 467 (June 2011) hereby incorporated by reference in its entirety. Within bacteria, the Type II effector system consists of a long pre-crRNA transcribed from the spacer-containing CRISPR locus, the multifunctional Cas9 protein, and a tracrRNA important for gRNA processing. The tracrRNAs hybridize to the repeat regions separating the spacers of the pre-crRNA, initiating dsRNA cleavage by endogenous RNase III, which is followed by a second cleavage event within each spacer by Cas9, producing mature crRNAs that remain associated with the tracrRNA and Cas9. TracrRNA-crRNA fusions are contemplated for use in the present methods.

According to one aspect, the enzyme of the present disclosure, such as Cas9 unwinds the DNA duplex and searches for sequences matching the crRNA to cleave. Target recognition occurs upon detection of complementarity between a “protospacer” sequence in the target DNA and the remaining spacer sequence in the crRNA. Importantly, Cas9 cuts the DNA only if a correct protospacer-adjacent motif (PAM) is also present at the 3′ end. According to certain aspects, different protospacer-adjacent motif can be utilized. For example, the S. pyogenes system requires an NGG sequence, where N can be any nucleotide. S. thermophilus Type II systems require NGGNG (see P. Horvath, R. Barrangou, CRISPR/Cas, the immune system of bacteria and archaea. Science 327, 167 (Jan. 8, 2010) hereby incorporated by reference in its entirety and NNAGAAW (see H. Deveau et al., Phage response to CRISPR-encoded resistance in Streptococcus thermophilus. Journal of bacteriology 190, 1390 (February 2008) hereby incorporated by reference in its entirety), respectively, while different S. mutans systems tolerate NGG or NAAR (see J. R. van der Ploeg, Analysis of CRISPR in Streptococcus mutans suggests frequent occurrence of acquired immunity against infection by M102-like bacteriophages. Microbiology 155, 1966 (June 2009) hereby incorporated by reference in its entirety. Bioinformatic analyses have generated extensive databases of CRISPR loci in a variety of bacteria that may serve to identify additional useful PAMs and expand the set of CRISPR-targetable sequences (see M. Rho, Y. W. Wu, H. Tang, T. G. Doak, Y. Ye, Diverse CRISPRs evolving in human microbiomes. PLoS genetics 8, e1002441 (2012) and D. T. Pride et al., Analysis of streptococcal CRISPRs from human saliva reveals substantial sequence diversity within and between subjects over time. Genome research 21, 126 (January 2011) each of which are hereby incorporated by reference in their entireties.

In S. pyogenes, Cas9 generates a blunt-ended double-stranded break 3bp upstream of the protospacer-adjacent motif (PAM) via a process mediated by two catalytic domains in the protein: an HNH domain that cleaves the complementary strand of the DNA and a RuvC-like domain that cleaves the non-complementary strand. See Jinek et al., Science 337, 816-821 (2012) hereby incorporated by reference in its entirety. Cas9 proteins are known to exist in many Type II CRISPR systems including the following as identified in the supplementary information to Makarova et al., Nature Reviews, Microbiology, Vol. 9, June 2011, pp. 467-477: Methanococcus maripaludis C7; Corynebacterium diphtheriae; Corynebacterium efficiens YS-314; Corynebacterium glutamicum ATCC 13032 Kitasato; Corynebacterium glutamicum ATCC 13032 Bielefeld; Corynebacterium glutamicum R; Corynebacterium kroppenstedtii DSM 44385; Mycobacterium abscessus ATCC 19977; Nocardia farcinica IFM10152; Rhodococcus erythropolis PR4; Rhodococcus jostii RHA1; Rhodococcus opacus B4 uid36573; Acidothermus cellulolyticus 11B; Arthrobacter chlorophenolicus A6; Kribbella flavida DSM 17836 uid43465; Thermomonospora curvata DSM 43183; Bifidobacterium dentium Bd1; Bifidobacterium longum DJO10A; Slackia heliotrinireducens DSM 20476; Persephonella marina EX H1; Bacteroides fragilis NCTC 9434; Capnocytophaga ochracea DSM 7271; Flavobacterium psychrophilum JIP02 86; Akkermansia muciniphila ATCC BAA 835; Roseiflexus castenholzii DSM 13941; Roseiflexus RS1; Synechocystis PCC6803; Elusimicrobium minutum Pei191; uncultured Termite group 1 bacterium phylotype Rs D17; Fibrobacter succinogenes S85; Bacillus cereus ATCC 10987; Listeria innocua; Lactobacillus casei; Lactobacillus rhamnosus GG; Lactobacillus salivarius UCC118; Streptococcus agalactiae A909; Streptococcus agalactiae NEM316; Streptococcus agalactiae 2603; Streptococcus dysgalactiae equisimilis GGS 124; Streptococcus equi zooepidemicus MGCS10565; Streptococcus gallolyticus UCN34 uid46061; Streptococcus gordonii Challis subst CH1; Streptococcus mutans NN2025 uid46353; Streptococcus mutans; Streptococcus pyogenes M1 GAS; Streptococcus pyogenes MGAS5005; Streptococcus pyogenes MGAS2096; Streptococcus pyogenes MGAS9429; Streptococcus pyogenes MGAS10270; Streptococcus pyogenes MGAS6180; Streptococcus pyogenes MGAS315; Streptococcus pyogenes SSI-1; Streptococcus pyogenes MGAS10750; Streptococcus pyogenes NZ 131; Streptococcus thermophiles CNRZ1066; Streptococcus thermophiles LMD-9; Streptococcus thermophiles LMG 18311; Clostridium botulinum A3 Loch Maree; Clostridium botulinum B Eklund 17B; Clostridium botulinum Ba4 657; Clostridium botulinum F Langeland; Clostridium cellulolyticum H10; Finegoldia magna ATCC 29328; Eubacterium rectale ATCC 33656; Mycoplasma gallisepticum; Mycoplasma mobile 163K; Mycoplasma penetrans; Mycoplasma synoviae 53; Streptobacillus moniliformis DSM 12112; Bradyrhizobium BTAil; Nitrobacter hamburgensis X14; Rhodopseudomonas palustris BisB18; Rhodopseudomonas palustris BisB5; Parvibaculum lavamentivorans DS-1; Dinoroseobacter shibae DFL 12; Gluconacetobacter diazotrophicus Pal 5 FAPERJ; Gluconacetobacter diazotrophicus Pal 5 JGI; Azospirillum B510 uid46085; Rhodospirillum rubrum ATCC 11170; Diaphorobacter TPSY uid29975; Verminephrobacter eiseniae EF01-2; Neisseria meningitides 053442; Neisseria meningitides alpha14; Neisseria meningitides Z2491; Desulfovibrio salexigens DSM 2638; Campylobacter jejuni doylei 269 97; Campylobacter jejuni 81116; Campylobacter jejuni; Campylobacter lari RM2100; Helicobacter hepaticus; Wolinella succinogenes; Tolumonas auensis DSM 9187; Pseudoalteromonas atlantica T6c; Shewanella pealeana ATCC 700345; Legionella pneumophila Paris; Actinobacillus succinogenes 130Z; Pasteurella multocida; Francisella tularensis novicida U112; Francisella tularensis holarctica; Francisella tularensis FSC 198; Francisella tularensis tularensis; Francisella tularensis WY96-3418; and Treponema denticola ATCC 35405. The Cas9 protein may be referred by one of skill in the art in the literature as Csn1. An exemplary S. pyogenes Cas9 protein sequence is provided in Deltcheva et al., Nature 471, 602-607 (2011) hereby incorporated by reference in its entirety.

Modification to the Cas9 protein is a representative embodiment of the present disclosure. CRISPR systems useful in the present disclosure are described in R. Barrangou, P. Horvath, CRISPR: new horizons in phage resistance and strain identification. Annual review of food science and technology 3, 143 (2012) and B. Wiedenheft, S. H. Sternberg, J. A. Doudna, RNA-guided genetic silencing systems in bacteria and archaea. Nature 482, 331 (Feb. 16, 2012) each of which are hereby incorporated by reference in their entireties.

According to certain aspects, the DNA binding protein is altered or otherwise modified to inactivate the nuclease activity. Such alteration or modification includes altering one or more amino acids to inactivate the nuclease activity or the nuclease domain. Such modification includes removing the polypeptide sequence or polypeptide sequences exhibiting nuclease activity, i.e. the nuclease domain, such that the polypeptide sequence or polypeptide sequences exhibiting nuclease activity, i.e. nuclease domain, are absent from the DNA binding protein. Other modifications to inactivate nuclease activity will be readily apparent to one of skill in the art based on the present disclosure. Accordingly, a nuclease-null DNA binding protein includes polypeptide sequences modified to inactivate nuclease activity or removal of a polypeptide sequence or sequences to inactivate nuclease activity. The nuclease-null DNA binding protein retains the ability to bind to DNA even though the nuclease activity has been inactivated. Accordingly, the DNA binding protein includes the polypeptide sequence or sequences required for DNA binding but may lack the one or more or all of the nuclease sequences exhibiting nuclease activity. Accordingly, the DNA binding protein includes the polypeptide sequence or sequences required for DNA binding but may have one or more or all of the nuclease sequences exhibiting nuclease activity inactivated.

According to one aspect, a DNA binding protein having two or more nuclease domains may be modified or altered to inactivate all but one of the nuclease domains. Such a modified or altered DNA binding protein is referred to as a DNA binding protein nickase, to the extent that the DNA binding protein cuts or nicks only one strand of double stranded DNA. When guided by RNA to DNA, the DNA binding protein nickase is referred to as an RNA guided DNA binding protein nickase. An exemplary DNA binding protein is an RNA guided DNA binding protein nuclease of a Type II CRISPR System, such as a Cas9 protein or modified Cas9 or homolog of Cas9. An exemplary DNA binding protein is a Cas9 protein nickase. An exemplary DNA binding protein is an RNA guided DNA binding protein of a Type II CRISPR System which lacks nuclease activity. An exemplary DNA binding protein is a nuclease-null or nuclease deficient Cas9 protein.

According to an additional aspect, nuclease-null Cas9 proteins are provided where one or more amino acids in Cas9 are altered or otherwise removed to provide nuclease-null Cas9 proteins. According to one aspect, the amino acids include D10 and H840. See Jinek et al., Science 337, 816-821 (2012). According to an additional aspect, the amino acids include D839 and N863. According to one aspect, one or more or all of D10, H840, D839 and H863 are substituted with an amino acid which reduces, substantially eliminates or eliminates nuclease activity. According to one aspect, one or more or all of D10, H840, D839 and H863 are substituted with alanine. According to one aspect, a Cas9 protein having one or more or all of D10, H840, D839 and H863 substituted with an amino acid which reduces, substantially eliminates or eliminates nuclease activity, such as alanine, is referred to as a nuclease-null Cas9 (“Cas9Nuc”) and exhibits reduced or eliminated nuclease activity, or nuclease activity is absent or substantially absent within levels of detection. According to this aspect, nuclease activity for a Cas9Nuc may be undetectable using known assays, i.e. below the level of detection of known assays.

According to one aspect, the Cas9 protein, Cas9 protein nickase or nuclease null Cas9 includes homologs and orthologs thereof which retain the ability of the protein to bind to the DNA and be guided by the RNA. According to one aspect, the Cas9 protein includes the sequence as set forth for naturally occurring Cas9 from S. thermophiles or S. pyogenes or S. aureus and protein sequences having at least 30%, 40%, 50%, 60%, 70%, 80%, 90%, 95%, 98% or 99% homology thereto and being a DNA binding protein, such as an RNA guided DNA binding protein.

An exemplary CRISPR system includes the S. thermophiles Cas9 nuclease (ST1 Cas9) (see Esvelt K M, et al., Orthogonal Cas9 proteins for RNA-guided gene regulation and editing, Nature Methods., (2013) hereby incorporated by reference in its entirety).An exemplary CRISPR system includes the S. pyogenes Cas9 nuclease (Sp. Cas9), an extremely high-affinity (see Sternberg, S. H., Redding, S., Jinek, M., Greene, E. C. & Doudna, J. A. DNA interrogation by the CRISPR RNA-guided endonuclease Cas9. Nature 507, 62-67 (2014) hereby incorporated by reference in its entirety), programmable DNA-binding protein isolated from a type II CRISPR-associated system (see Garneau, J. E. et al. The CRISPR/Cas bacterial immune system cleaves bacteriophage and plasmid DNA. Nature 468, 67-71 (2010) and Jinek, M. et al. A programmable dual-RNA-guided DNA endonuclease in adaptive bacterial immunity. Science 337, 816-821 (2012) each of which are hereby incorporated by reference in its entirety). According to certain aspects, a nuclease null or nuclease deficient Cas 9 can be used in the methods described herein. Such nuclease null or nuclease deficient Cas9 proteins are described in Gilbert, L. A. et al. CRISPR-mediated modular RNA-guided regulation of transcription in eukaryotes. Cell 154, 442-451 (2013); Mali, P. et al. CAS9 transcriptional activators for target specificity screening and paired nickases for cooperative genome engineering. Nature biotechnology 31, 833-838 (2013); Maeder, M. L. et al. CRISPR RNA-guided activation of endogenous human genes. Nature methods 10, 977-979 (2013); and Perez-Pinera, P. et al. RNA-guided gene activation by CRISPR-Cas9-based transcription factors. Nature methods 10, 973-976 (2013) each of which are hereby incorporated by reference in its entirety. The DNA locus targeted by Cas9 (and by its nuclease-deficient mutant, “dCas9” precedes a three nucleotide (nt) 5′-NGG-3′ “PAM” sequence, and matches a 15-22-nt guide or spacer sequence within a Cas9-bound RNA cofactor, referred to herein and in the art as a guide RNA. Altering this guide RNA is sufficient to target Cas9 or a nuclease deficient Cas9 to a target nucleic acid. In a multitude of CRISPR-based biotechnology applications (see Mali, P., Esvelt, K. M. & Church, G. M. Cas9 as a versatile tool for engineering biology. Nature methods 10, 957-963 (2013); Hsu, P. D., Lander, E. S. & Zhang, F. Development and Applications of CRISPR-Cas9 for Genome Engineering. Cell 157, 1262-1278 (2014); Chen, B. et al. Dynamic imaging of genomic loci in living human cells by an optimized CRISPR/Cas system. Cell 155, 1479-1491 (2013); Shalem, O. et al. Genome-scale CRISPR-Cas9 knockout screening in human cells. Science 343, 84-87 (2014); Wang, T., Wei, J. J., Sabatini, D. M. & Lander, E. S. Genetic screens in human cells using the CRISPR-Cas9 system. Science 343, 80-84 (2014); Nissim, L., Perli, S. D., Fridkin, A., Perez-Pinera, P. & Lu, T. K. Multiplexed and Programmable Regulation of Gene Networks with an Integrated RNA and CRISPR/Cas Toolkit in Human Cells. Molecular cell 54, 698-710 (2014); Ryan, O. W. et al. Selection of chromosomal DNA libraries using a multiplex CRISPR system. eLife 3 (2014); Gilbert, L. A. et al. Genome-Scale CRISPR-Mediated Control of Gene Repression and Activation. Cell (2014); and Citorik, R. J., Mimee, M. & Lu, T. K. Sequence-specific antimicrobials using efficiently delivered RNA-guided nucleases. Nature biotechnology (2014) each of which are hereby incorporated by reference in its entirety), the guide is often presented in a so-called sgRNA (single guide RNA), wherein the two natural Cas9 RNA cofactors (gRNA and tracrRNA) are fused via an engineered loop or linker.

According to one aspect, the Cas9 protein is an enzymatically active Cas9 protein, a Cas9 protein wild-type protein, a Cas9 protein nickase or a nuclease null or nuclease deficient Cas9 protein. Additional exemplary Cas9 proteins include Cas9 proteins attached to, bound to or fused with functional proteins such as transcriptional regulators, such as transcriptional activators or repressors, a Fok-domain, such as Fok 1, an aptamer, a binding protein, PP7, MS2 and the like.

The disclosure provides mutants of Cas proteins that improve binding to target nucleic acid sequences having an adjacent functional protospacer adjacent motif. The disclosure provides mutants of Cas proteins that allow binding to target nucleic acid sequences in the absence of an adjacent functional protospacer adjacent motif. The disclosure provides a mutant Cas protein, such as a mutant Cas9 protein, where one or more mutations alter the charge of the Cas protein compared to the wild type Cas protein. The disclosure provides a mutant Cas protein having an altered charge compared to the wild type Cas protein. The disclosure provides that the altered charge promotes binding of the Cas protein to a nucleic acid, such as DNA. The disclosure provides a mutant Cas protein having a lower negative charge compared to the wild type Cas protein. The disclosure provides a mutant Cas protein including one or more amino acid mutations from a negatively charged amino acid to a neutral charged amino acid or a positively charged amino acid. The disclosure provides a mutant Cas protein including one or more amino acid mutations from a neutral charged amino acid to a positively charged amino acid. The disclosure provides a mutant Cas protein including one or more amino acid mutations selected from the group consisting of a negatively charged amino acid to a neutral charged amino acid, a negatively charged amino acid to a positively charged amino acid and a neutral charged amino acid to a positively charged amino acid. The disclosure provides a mutant Cas protein having a lower electrostatic repulsion to DNA compared to wild type or unmutated Cas protein. The disclosure provides that mutant Cas proteins have one or more of the following mutations: G1104K, L1111H, D1135Y and N1317K.

According to certain aspects, the mutant Cas9 protein may be delivered directly to a cell by methods known to those of skill in the art, including injection or lipofection, or as translated from its cognate mRNA, or transcribed from its cognate DNA into mRNA (and thereafter translated into protein). Mutant Cas9 DNA and mRNA may be themselves introduced into cells through electroporation, transient and stable transfection (including lipofection) and viral transduction or other methods known to those of skill in the art.

### Accessory DNA Binding Proteins or Domains

The disclosure provides for the use of accessory DNA binding peptides or proteins or domains that may be fused to a Cas protein to assist the Cas protein with binding at a target nucleic acid sequence. Exemplary accessory DNA binding peptides, proteins or domains include sso7d, sssIM, DNAse I, sfGFP+15, micrococcal nuclease, tat peptide, ctb peptide and the like. It is to be understood that additional exemplary accessory DNA binding peptides, proteins or domains can be identified by those of skill in the art based on the present disclosure.

According to certain aspects, an accessory DNA binding protein or domain is altered or otherwise modified to inactivate enzymatic or other activity which may otherwise be associated with the accessory DNA binding protein or domain in the unaltered state. The disclosure provides that the accessory DNA binding protein or domain exhibits nucleic acid binding activity, but has no other substantial activity that would otherwise interfere with DNA binding activity or other substantial activity directed to the nucleic acid to which is binds. Such alteration or modification includes altering one or more amino acids to inactivate the undesired enzymatic activity present. Such modification includes removing the polypeptide sequence or polypeptide sequences exhibiting enzymatic activity, such that the polypeptide sequence or polypeptide sequences exhibiting enzymatic activity are absent from the DNA binding protein. Other modifications to inactivate enzymatic activity will be readily apparent to one of skill in the art based on the present disclosure. Accordingly, an enzymatic-null DNA binding protein or domain includes polypeptide sequences modified to inactivate enzymatic activity or removal of a polypeptide sequence or sequences to inactivate enzymatic activity. The enzymatic-null DNA binding protein or domain retains the ability to bind to DNA even though the enzymatic activity has been inactivated. Accordingly, the accessory DNA binding protein or domain includes the polypeptide sequence or sequences required for DNA binding but may lack the one or more or all of the sequences exhibiting enzymatic activity. Accordingly, the accessory DNA binding protein or domain includes the polypeptide sequence or sequences required for DNA binding but may have one or more or all of the sequences exhibiting enzymatic activity inactivated.

### Guide RNA Description

Embodiments of the present disclosure are directed to the use of a CRISPR/Cas system and, in particular, a guide RNA which may include one or more of a spacer sequence, a tracr mate sequence and a tracr sequence. The term spacer sequence is understood by those of skill in the art and may include any polynucleotide having sufficient complementarity with a target nucleic acid sequence to hybridize with the target nucleic acid sequence and direct sequence-specific binding of a CRISPR complex to the target sequence. The guide RNA may be formed from a spacer sequence covalently connected to a tracr mate sequence (which may be referred to as a crRNA) and a separate tracr sequence, wherein the tracr mate sequence is hybridized to a portion of the tracr sequence. According to certain aspects, the tracr mate sequence and the tracr sequence are connected or linked such as by covalent bonds by a linker sequence, which construct may be referred to as a fusion of the tracr mate sequence and the tracr sequence. The linker sequence referred to herein is a sequence of nucleotides, referred to herein as a nucleic acid sequence, which connect the tracr mate sequence and the tracr sequence. Accordingly, a guide RNA may be a two component species (i.e., separate crRNA and tracr RNA which hybridize together) or a unimolecular species (i.e., a crRNA-tracr RNA fusion, often termed an sgRNA).

According to certain aspects, the guide RNA is between about 10 to about 500 nucleotides. According to one aspect, the guide RNA is between about 20 to about 100 nucleotides. According to certain aspects, the spacer sequence is between about 10 and about 500 nucleotides in length. According to certain aspects, the tracr mate sequence is between about 10 and about 500 nucleotides in length. According to certain aspects, the tracr sequence is between about 10 and about 100 nucleotides in length. According to certain aspects, the linker nucleic acid sequence is between about 10 and about 100 nucleotides in length.

According to one aspect, embodiments described herein include guide RNA having a length including the sum of the lengths of a spacer sequence, tracr mate sequence, tracr sequence, and linker sequence (if present). Accordingly, such a guide RNA may be described by its total length which is a sum of its spacer sequence, tracr mate sequence, tracr sequence, and linker sequence (if present). According to this aspect, all of the ranges for the spacer sequence, tracr mate sequence, tracr sequence, and linker sequence (if present) are incorporated herein by reference and need not be repeated. A guide RNA as described herein may have a total length based on summing values provided by the ranges described herein. Aspects of the present disclosure are directed to methods of making such guide RNAs as described herein by expressing constructs encoding such guide RNA using promoters and terminators and optionally other genetic elements as described herein.

According to certain aspects, the guide RNA may be delivered directly to a cell as a native species by methods known to those of skill in the art, including injection or lipofection, or as transcribed from its cognate DNA, with the cognate DNA introduced into cells through electroporation, transient and stable transfection (including lipofection) and viral transduction.

### Donor Description

The term “donor nucleic acid” include a nucleic acid sequence which is to be inserted into mitochondrial DNA according to methods described herein for expression by the mitochondrial DNA. The donor nucleic acid sequence may be expressed by the cell.

According to one aspect, the donor nucleic acid is exogenous to the cell. According to one aspect, the donor nucleic acid is foreign to the cell. According to one aspect, the donor nucleic acid is non-naturally occurring within the cell.

### Transcription Regulator Description

According to one aspect, an engineered Cas9-gRNA system is provided which enables RNA-guided DNA regulation in cells such as human cells by tethering transcriptional activation domains to either a nuclease-null Cas9 or to guide RNAs. According to one aspect of the present disclosure, one or more transcriptional regulatory proteins or domains (such terms are used interchangeably) are joined or otherwise connected to a nuclease-deficient Cas9 or one or more guide RNA (gRNA). The transcriptional regulatory domains correspond to targeted loci. Accordingly, aspects of the present disclosure include methods and materials for localizing transcriptional regulatory domains to targeted loci by fusing, connecting or joining such domains to either Cas9N or to the gRNA.

According to one aspect, a mutant Cas9N-fusion protein capable of transcriptional activation is provided. According to one aspect, a VP64 activation domain (see Zhang et al., Nature Biotechnology 29, 149-153 (2011) hereby incorporated by reference in its entirety) is joined, fused, connected or otherwise tethered to the C. terminus of mutant Cas9N. According to one method, the transcriptional regulatory domain is provided to the site of target mitochondrial DNA by the mutant Cas9N protein. According to one method, a mutant Cas9N fused to a transcriptional regulatory domain is provided within a cell along with one or more guide RNAs. The mutant Cas9N with the transcriptional regulatory domain fused thereto bind at or near target mitochondrial DNA. The one or more guide RNAs bind at or near target mitochondrial DNA. The transcriptional regulatory domain regulates expression of the target mitochondrial nucleic acid sequence. According to a specific aspect, a mutant Cas9N-VP64 fusion activated transcription of reporter constructs when combined with gRNAs targeting sequences near the promoter, thereby displaying RNA-guided transcriptional activation.

According to one aspect, a gRNA-fusion protein capable of transcriptional activation is provided. According to one aspect, a VP64 activation domain is joined, fused, connected or otherwise tethered to the gRNA. According to one method, the transcriptional regulatory domain is provided to the site of target mitochondrial DNA by the gRNA. According to one method, a gRNA fused to a transcriptional regulatory domain is provided within a cell along with a mutant Cas9N protein. The mutant Cas9N binds at or near target DNA. The one or more guide RNAs with the transcriptional regulatory protein or domain fused thereto bind at or near target DNA. The transcriptional regulatory domain regulates expression of the target gene. According to a specific aspect, a mutant Cas9N protein and a gRNA fused with a transcriptional regulatory domain activated transcription of reporter constructs, thereby displaying RNA-guided transcriptional activation.

Transcriptional regulator proteins or domains which are transcriptional activators include VP16 and VP64 and others readily identifiable by those skilled in the art based on the present disclosure.

### Target Nucleic Acid

Target nucleic acids include any nucleic acid sequence to which a co-localization complex as described herein can be useful to either cut, nick or regulate. Target nucleic acids include nucleic acid sequences, such as genomic nucleic acids, such as genes, capable of being expressed into proteins. For purposes of the present disclosure, a co-localization complex can bind to or otherwise co-localize with the target nucleic acid at or adjacent or near the target nucleic acid and in a manner in which the co-localization complex may have a desired effect on the target nucleic acid. One of skill based on the present disclosure will readily be able to identify or design guide RNAs and Cas9 proteins which co-localize to a target nucleic acid. One of skill will further be able to identify transcriptional regulator proteins or domains which likewise co-localize to a target nucleic acid.

### Foreign Nucleic Acids Description

Foreign nucleic acids (i.e. those which are not part of a cell's natural nucleic acid composition) may be introduced into a cell using any method known to those skilled in the art for such introduction. Such methods include transfection, transduction, viral transduction, microinjection, lipofection, nucleofection, nanoparticle bombardment, transformation, conjugation and the like. One of skill in the art will readily understand and adapt such methods using readily identifiable literature sources.

### Cells

Cells according to the present disclosure include any cell into which foreign nucleic acids can be introduced and expressed as described herein. It is to be understood that the basic concepts of the present disclosure described herein are not limited by cell type. Cells according to the present disclosure include eukaryotic cells, prokaryotic cells, animal cells, plant cells, fungal cells, archael cells, eubacterial cells and the like. Cells include eukaryotic cells such as yeast cells, plant cells, and animal cells. Particular cells include mammalian cells. Further, cells include any in which it would be beneficial or desirable to cut, nick or regulate a target nucleic acid. Such cells may include those which are deficient in expression of a particular protein leading to a disease or detrimental condition. Such diseases or detrimental conditions are readily known to those of skill in the art. According to the present disclosure, the nucleic acid responsible for expressing the particular protein may be targeted by the methods described herein and a transcriptional activator resulting in upregulation of the target nucleic acid and corresponding expression of the particular protein. In this manner, the methods described herein provide therapeutic treatment. Such cells may include those which are over express a particular protein leading to a disease or detrimental condition. Such diseases or detrimental conditions are readily known to those of skill in the art. According to the present disclosure, the nucleic acid responsible for expressing the particular protein may be targeted by the methods described herein and a transcriptional depressor or repressor resulting in downregulation of the target nucleic acid and corresponding expression of the particular protein. In this manner, the methods described herein provide therapeutic treatment.

According to one aspect, the cell is a eukaryotic cell. According to one aspect, the cell is a yeast cell, a plant cell or an animal cell. According to one aspect, the cell is a mammalian cell. According to one aspect, the cell is a human cell. According to one aspect, the cell is a stem cell whether adult or embryonic. According to one aspect, the cell is a pluripotent stem cell. According to one aspect, the cell is an induced pluripotent stem cell. According to one aspect, the cell is a human induced pluripotent stem cell.

### Vectors

Vectors are contemplated for use with the methods and constructs described herein. The term “vector” includes a nucleic acid molecule capable of transporting another nucleic acid to which it has been linked. Vectors used to deliver the nucleic acids to cells as described herein include vectors known to those of skill in the art and used for such purposes. Certain exemplary vectors may be plasmids, lentiviruses or adeno-associated viruses known to those of skill in the art. Vectors include, but are not limited to, nucleic acid molecules that are single-stranded, doublestranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, lentiviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Common expression vectors of utility in recombinant DNA techniques are often in the form of plasmids. Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

Methods of non-viral delivery of nucleic acids or native DNA binding protein, native guide RNA or other native species include lipofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration). The term native includes the protein, enzyme or guide RNA species itself and not the nucleic acid encoding the species. According to certain aspects, the vectors are engineered to specifically target to mitochondria and/or codon optimized for mitochondrial specific delivery of the nucleic acid sequences within the vectors.

### Regulatory Elements and Terminators and Tags

Regulatory elements are contemplated for use with the methods and constructs described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector may comprise one or more pol III promoter (e.g. 1, 2, 3, 4, 5, or more pol III promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter and Pol II promoters described herein. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

Aspects of the methods described herein may make use of terminator sequences. A terminator sequence includes a section of nucleic acid sequence that marks the end of a gene or operon in genomic DNA during transcription. This sequence mediates transcriptional termination by providing signals in the newly synthesized mRNA that trigger processes which release the mRNA from the transcriptional complex. These processes include the direct interaction of the mRNA secondary structure with the complex and/or the indirect activities of recruited termination factors. Release of the transcriptional complex frees RNA polymerase and related transcriptional machinery to begin transcription of new mRNAs. Terminator sequences include those known in the art and identified and described herein.

Aspects of the methods described herein may make use of epitope tags and reporter gene sequences. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-S-transferase (GST), horseradish peroxidase (HRP), chloramphenicol acetyltransferase (CAT) beta-galactosidase, betaglucuronidase, luciferase, green fluorescent protein (GFP), HcRed, DsRed, cyan fluorescent protein (CFP), yellow fluorescent protein (YFP), and autofluorescent proteins including blue fluorescent protein (BFP).

### Delivery Description

Embodiments of the present disclosure are directed to a method of delivering a mutant Cas9 protein to cells within a subject comprising administering to the subject, such as systemically administering to the subject, such as by intravenous administration or injection, intraperitoneal administration or injection, intramuscular administration or injection, intracranial administration or injection, intraocular administration or injection, subcutaneous administration or injection, a mutant Cas9 protein or a nucleic acid encoding the mutant Cas9 protein.

Embodiments of the present disclosure are directed to a method of delivering a guide RNA to cells within a subject comprising administering to the subject, such as systemically administering to the subject, such as by intravenous administration or injection, intraperitoneal administration or injection, intramuscular administration or injection, intracranial administration or injection, intraocular administration or injection, subcutaneous administration or injection, a guide RNA or a nucleic acid encoding the guide RNA.

Embodiments of the present disclosure are directed to a method of delivering a mutant Cas9 protein and a guide RNA to cells within a subject comprising administering to the subject, such as systemically administering to the subject, such as by intravenous administration or injection, intraperitoneal administration or injection, intramuscular administration or injection, intracranial administration or injection, intraocular administration or injection, subcutaneous administration or injection, a mutant Cas9 protein or a nucleic acid encoding the mutant Cas9 protein and a guide RNA or a nucleic acid encoding the guide RNA.

The following examples are set forth as being representative of the present disclosure. These examples are not to be construed as limiting the scope of the present disclosure as these and other equivalent embodiments will be apparent in view of the present disclosure, figures and accompanying claims.

### EXAMPLE I

### Materials and Methods

### Vector Design and Construction

Cas9 expression plasmids are based off of vectors Cas9 (Addgene #41815) and Cas9-m4 (Addgene #47316). Fusions between DNA binding domains and Cas9 were made by using golden gate compatible Cas9 cloning plasmids and appending the appropriate AF (TTTTGCTCTTCTAGTGGCGGGTCAGGGTCG) (SEQ ID NO:1) and bbR (TTTTGCTCTTCTCTA) (SEQ ID NO:2) sequence to the 5′ and 3′ ends of the DNA binding domain to be inserted, respectively. MS2-p65-hsf1 expression construct is previously published (Addgene #61426) (PMID: 25494202) . Activation reporter constructs are based on a previous construct (Addgene #47320) with minor modifications, where indicated the downstream fluorescent protein was changed to iRFP713 or the sequence of the PAM was altered from NGG to NAG, NGA or NGC. Reporters for cutting and repression are previously described (PMID: 26344044). Sequences for golden gate compatible Cas9 vectors and gRNA cloning vector along with the amino acid sequence of the utilized DNA binding domains are provided within Supplementary Sequences.

### Golden Gate Cloning

40 pmoles of Cas9 golden gate compatible vector and 40 pmoles of insert were mixed in a 20 ul reaction containing 2 ul of cutsmart buffer (NEB), 1 ul SapI enzyme (NEB), 2 ul ATP, 1 ul T4 DNA ligase (NEB) and were placed in a thermocyclers for 2.5 hours at 37° C. followed by heat inactivation at 65° C. for 20 minutes and then 80° C. for 10 minutes. An additional 1 ul of SapI enzyme was then added to each reaction and allowed to digest at 37° C. for an additional hour. Golden gate reactions were transformed into DH5alpha chemically competent E. coli.

### Mammalian Cell Culture

All cell culture experiments were performed in HEK293T cells (gift from P. Mali, UCSD, San Diego, Calif.). Cells were maintained in Dulbecco's Modified Eagle Medium supplemented with 10% heat inactivated FBS and penicillin-streptomycin (cell culture materials were purchased from ThermoFisher) and were maintained in an incubator at 5% CO₂ and 37° C. Cells were passaged every 3-4 days upon reaching confluency and were seeded into 24-well plates the day before transfection.

### Activation, Repression and Cutting Assays

To detect increases in gene activation with both canonical PAMs (NGG) and non-canonical PAMs (NAG, NGA, and NGC) a tdtomato reporter construct containing a minimal CMV promoter and a Cas9 binding site upstream of a fluorescent protein (iRFP713 for NGG PAM or tdtomato for NAG, NGA, and NGC PAMs) was employed. The given Cas9 variant either a point mutant or fused to a DNA binding peptide/DNA binding protein/DNA binding domain was directed to the activation reporter construct using a gRNA containing several MS2 hairpins, allowing the recruitment of an MS2 binding protein-p65-hsf1 activation domain to the site of Cas9 binding. For experiments involving nuclease competent Cas9. 14nt gRNAs were used. In cases where nuclease null dCas9 were employed, 20nt gRNAs were used. In cases where repression was measured, nuclease null Cas9 or nuclease null Cas9 fused to a DNA binding peptide/protein was directed to a YFP reporter construct that was activated by a GAL4-VP16 fusion. For all fluorescent reporter assays, cell were also co-transfected with a plasmid expressing EBFP enabling selection of transfected cells by gating our analysis on cells with >10\hat{ }3 arbitrary fluorescent units of EBFP expression. For experiments involving deletion (cutting), the reporter plasmid was transfected along with the Cas9 protein of interest and gRNA targeting the reporter, no EBFP plasmid was co-transfected.

### Transfections

For each well to be transfected, 200 ng of a given Cas9 component was utilized along with 10 ng of the required gRNA and 60 ng of the reporter construct for activation or cutting. In cases of repression 50 ng of the required reporter construct for repression, plus 50 ng of the needed Ga14-VP16 activator plasmid were used. For experiments involving non-canonical PAM experiments, a mixture of 3 different reporter plasmids each at 20 ng was provided. For experiments involving endogenous gene activation, both TTN and ASCL1 were simultaneously targeted by co-transfecting cells with 10 ng of each of the respective targeting gRNAs along with the designated Cas9 protein.

Lipofectamine 2000 (ThermoFisher) was utilized for transfecting HEK293T cells and was performed per the manufacturer's instructions. The day after transfection, the media was replaced and cells were analyzed 48-72 hours post transfection.

RNA Extraction and qPCR Analysis for Mammalian Cell Lines

When harvesting cells for RNA analysis, the RNAeasy Plus Mini Kit (Qiagen) was employed. cDNA was generated using the iScript cDNA synthesis kit (Bio-Rad) with 500 ng of input RNA provided per reaction. For qPCR analysis the KAPA SYBR Fast Universal 2× quantitative PCR kit (Kapa Biosystems) was employed with 0.5 ul of input cDNA from the previous reverse transcription reaction used per reaction (total reaction volume 20 ul). The ACTB gene was used for internal sample normalization.

Supplemental Sequences:
Golden gate compatible Cas9 plasmid sequences and gRNA expression vector:
dCas9-m4-golden gate compatible vector (SEQ ID NO: 3):
gttaggcgttttgcgctgcttcgcgatgtacgggccagatatacgcgttgacattgattattgactagttattaatagtaatcaattacgggg
tcattagttcatagcccatatatggagttccgcgttacataacttacggtaaatggcccgcctggctgaccgcccaacgacccccgccc
attgacgtcaataatgacgtatgttcccatagtaacgccaatagggactttccattgacgtcaatgggtggagtatttacggtaaactgcc
cacttggcagtacatcaagtgtatcatatgccaagtacgccccctattgacgtcaatgacggtaaatggcccgcctggcattatgcccag
tacatgaccttatgggactttcctacttggcagtacatctacgtattagtcatcgctattaccatggtgatgcggttttggcagtacatcaatg
ggcgtggatagcggtttgactcacggggatttccaagtctccaccccattgacgtcaatgggagtttgttttggcaccaaaatcaacggg
actttccaaaatgtcgtaacaactccgccccattgacgcaaatgggcggtaggcgtgtacggtgggaggtctatataagcagagctcgt
ttagtgaaccgtcagatcgcctggagacgccatccacgctgttttgacctccatagaagacaccgggaccgatccagcctccggactc
tagaggatcgaaccottgccaccATGGACAAGAAGTACTCCATTGGGCTCGCTATCGGCACAA
ACAGCGTCGGCTGGGCCGTCATTACGGACGAGTACAAGGTGCCGAGCAAAAAAT
TCAAAGTTCTGGGCAATACCGATCGCCACAGCATAAAGAAGAACCTCATTGGCG
CCCTCCTGTTCGACTCCGGGGAGACGGCCGAAGCCACGCGGCTCAAAAGAACAG
CACGGCGCAGATATACCCGCAGAAAGAATCGGATCTGCTACCTGCAGGAGATCT
TTAGTAATGAGATGGCTAAGGTGGATGACTCTTTCTTCCATAGGCTGGAGGAGTC
CTTTTTGGTGGAGGAGGATAAAAAGCACGAGCGCCACCCAATCTTTGGCAATAT
CGTGGACGAGGTGGCGTACCATGAAAAGTACCCAACCATATATCATCTGAGGAA
GAAGCTTGTAGACAGTACTGATAAGGCTGACTTGCGGTTGATCTATCTCGCGCTG
GCGCATATGATCAAATTTCGGGGACACTTCCTCATCGAGGGGGACCTGAACCCA
GACAACAGCGATGTCGAtAAACTCTTTATCCAACTGGTTCAGACTTACAATCAGC
TTTTCGAAGAGAACCCGATCAACGCATCCGGAGTTGACGCCAAAGCAATCCTGA
GCGCTAGGCTGTCCAAATCCCGGCGGCTCGAAAACCTCATCGCACAGCTCCCTGG
GGAGAAGAAGAACGGCCTGTTTGGTAATCTTATCGCCCTGTCACTCGGGCTGACC
CCCAACTTTAAATCTAACTTCGACCTGGCCGAAGATGCCAAGCTTCAACTGAGCA
AAGACACCTACGATGATGATCTCGACAATCTGCTGGCCCAGATCGGCGACCAGT
ACGCAGACCTTTTTTTGGCGGCAAAGAACCTGTCAGACGCCATTCTGCTGAGTGA
TATTCTGCGAGTGAACACGGAGATCACCAAAGCTCCGCTGAGCGCTAGTATGAT
CAAGCGCTATGATGAGCACCACCAAGACTTGACTTTGCTGAAGGCCCTTGTCAGA
CAGCAACTGCCTGAGAAGTACAAGGAAATTTTCTTCGATCAGTCTAAAAATGGCT
ACGCCGGATACATTGACGGCGGAGCAAGCCAGGAGGAATTTTACAAATTTATTA
AGCCCATCTTGGAAAAAATGGACGGCACCGAGGAGCTGCTGGTAAAGCTTAACA
GAGAAGATCTGTTGCGCAAACAGCGCACTTTCGACAATGGAAGCATCCCCCACC
AGATTCACCTGGGCGAACTGCACGCTATCCTCAGGCGGCAAGAGGATTTCTACCC
CTTTTTGAAAGATAACAGGGAAAAGATTGAGAAAATCCTCACATTTCGGATACC
CTACTATGTAGGCCCCCTCGCCCGGGGAAATTCCAGATTCGCGTGGATGACTCGC
AAATCAGAAGAGACCATCACTCCCTGGAACTTCGAGGAAGTCGTGGATAAGGGG
GCCTCTGCCCAGTCCTTCATCGAAAGGATGACTAACTTTGATAAAAATCTGCCTA
ACGAAAAGGTGCTTCCTAAACACTCTCTGCTGTACGAGTACTTCACAGTTTATAA
CGAGCTCACCAAGGTCAAATACGTCACAGAAGGGATGAGAAAGCCAGCATTCCT
GTCTGGAGAGCAGAAGAAAGCTATCGTGGACCTCCTCTTCAAGACGAACCGGAA
AGTTACCGTGAAACAGCTCAAAGAAGACTATTTCAAAAAGATTGAATGTTTCGA
CTCTGTTGAAATCAGCGGAGTGGAGGATCGCTTCAACGCATCCCTGGGAACGTAT
CACGATCTCCTGAAAATCATTAAAGACAAGGACTTCCTGGACAATGAGGAGAAC
GAGGACATTCTTGAGGACATTGTCCTCACCCTTACGTTGTTTGAAGATAGGGAGA
TGATTGAAGAACGCTTGAAAACTTACGCTCATCTCTTCGACGACAAAGTCATGAA
ACAGCTCAAGAGGCGCCGATATACAGGATGGGGGCGGCTGTCAAGAAAACTGAT
CAATGGGATCCGAGACAAGCAGAGTGGAAAGACAATCCTGGATTTTCTTAAGTC
CGATGGATTTGCCAACCGGAACTTCATGCAGTTGATCCATGATGACTCTCTCACC
TTTAAGGAGGACATCCAGAAAGCACAAGTTTCTGGCCAGGGGGACAGTCTTCAC
GAGCACATCGCTAATCTTGCAGGTAGCCCAGCTATCAAAAAGGGAATACTGCAG
ACCGTTAAGGTCGTGGATGAACTCGTCAAAGTAATGGGAAGGCATAAGCCCGAG
AATATCGTTATCGAGATGGCCCGAGAGAACCAAACTACCCAGAAGGGACAGAAG
AACAGTAGGGAAAGGATGAAGAGGATTGAAGAGGGTATAAAAGAACTGGGGTC
CCAAATCCTTAAGGAACACCCAGTTGAAAACACCCAGCTTCAGAATGAGAAGCT
CTACCTGTACTACCTGCAGAACGGCAGGGACATGTACGTGGATCAGGAACTGGA
CATCAATCGGCTCTCCGACTACGACGTGGCTGCTATCGTGCCCCAGTCTTTTCTCA
AAGATGATTCTATTGATAATAAAGTGTTGACAAGATCCGATAAAgcTAGAGGGAA
GAGTGATAACGTCCCCTCAGAAGAAGTTGTCAAGAAAATGAAAAATTATTGGCG
GCAGCTGCTGAACGCCAAACTGATCACACAACGGAAGTTCGATAATCTGACTAA
GGCTGAACGAGGTGGCCTGTCTGAGTTGGATAAAGCCGGCTTCATCAAAAGGCA
GCTTGTTGAGACACGCCAGATCACCAAGCACGTGGCCCAAATTCTCGATTCACGC
ATGAACACCAAGTACGATGAAAATGACAAACTGATTCGAGAGGTGAAAGTTATT
ACTCTGAAGTCTAAGCTGGTCTCAGATTTCAGAAAGGACTTTCAGTTTTATAAGG
TGAGAGAGATCAACAATTACCACCATGCGCATGATGCCTACCTGAATGCAGTGG
TAGGCACTGCACTTATCAAAAAATATCCCAAGCTTGAATCTGAATTTGTTTACGG
AGACTATAAAGTGTACGATGTTAGGAAAATGATCGCAAAGTCTGAGCAGGAAAT
AGGCAAGGCCACCGCTAAGTACTTCTTTTACAGCAATATTATGAATTTTTTCAAG
ACCGAGATTACACTGGCCAATGGAGAGATTCGGAAGCGACCACTTATCGAAACA
AACGGAGAAACAGGAGAAATCGTGTGGGACAAGGGTAGGGATTTCGCGACAGT
CCGGAAGGTCCTGTCCATGCCGCAGGTGAACATCGTTAAAAAGACCGAAGTACA
GACCGGAGGCTTCTCCAAGGAAAGTATCCTCCCGAAAAGGAACAGCGACAAGCT
GATCGCACGCAAAAAAGATTGGGACCCCAAGAAATACGGCGGATTCGATTCTCC
TACAGTCGCTTACAGTGTACTGGTTGTGGCCAAAGTGGAGAAAGGGAAGTCTAA
AAAACTCAAAAGCGTCAAGGAACTGCTGGGCATCACAATCATGGAGCGATCAAG
CTTCGAAAAAAACCCCATCGACTTTCTCGAGGCGAAAGGATATAAAGAGGTCAA
AAAAGACCTCATCATTAAGCTTCCCAAGTACTCTCTCTTTGAGCTTGAAAACGGC
CGGAAACGAATGCTCGCTAGTGCGGGCGAGCTGCAGAAAGGTAACGAGCTGGCA
CTGCCCTCTAAATACGTTAATTTCTTGTATCTGGCCAGCCACTATGAAAAGCTCA
AAGGGTCTCCCGAAGATAATGAGCAGAAGCAGCTGTTCGTGGAACAACACAAAC
ACTACCTTGATGAGATCATCGAGCAAATAAGCGAATTCTCCAAAAGAGTGATCC
TCGCCGACGCTAACCTCGATAAGGTGCTTTCTGCTTACAATAAGCACAGGGATAA
GCCCATCAGGGAGCAGGCAGAAAACATTATCCACTTGTTTACTCTGACCAACTTG
GGCGCGCCTGCAGCCTTCAAGTACTTCGACACCACCATAGACAGAAAGCGGTAC
ACCTCTACAAAGGAGGTCCTGGACGCCACACTGATTCATCAGTCAATTACGGGG
CTCTATGAAACAAGAATCGACCTCTCTCAGCTCGGTGGAGACAGCAGGGCTGAC
CCCAAGAAGAAGAGGAAGGTGagtggtggaggaagttgaagagctatgtttagatatccaaaccaggctcttctta
gaagaattcgatccctaccggttagtaatgagtttaaacgggggaggctaactgaaacacggaaggagacaataccggaaggaacc
cgcgctatgacggcaataaaaagacagaataaaacgcacgggtgttgggtcgtttgttcataaacgcggggttcggtcccagggctg
gcactctgtcgataccccaccgagaccccattggggccaatacgcccgcgtttcttccttttccccaccccaccccccaagttcgggtg
aaggcccagggctcgcagccaacgtcggggcggcaggccctccatagtcggtcgttcggctgcggcgagcggtatcagctcactc
aaaggcggtaatacggttatccacagaatcaggggataacgcaggaaagaacatgtgagcaaaaggccagcaaaaggccaggaa
ccgtaaaaaggccgcgttgctggcgtttttccataggctccgcccccctgacgagcatcacaaaaatcgacgctcaagtcagaggtgg
cgaaacccgacaggactataaagataccaggcgtttccccctggaagctccctcgtgcgctctcctgttccgaccctgccgcttaccg
gatacctgtccgcctttctcccttcgggaagcgtggcgctttctcatagctcacgctgtaggtatctcagttcggtgtaggtcgttcgctcc
aagctgggctgtgtgcacgaaccccccgttcagcccgaccgctgcgccttatccggtaactatcgtcttgagtccaacccggtaagac
acgacttatcgccactggcagcagccactggtaacaggattagcagagcgaggtatgtaggcggtgctacagagttcttgaagtggtg
gcctaactacggctacactagaaggacagtatttggtatctgcgctctgctgaagccagttaccttcggaaaaagagttggtagctcttg
atccggcaaacaaaccaccgctggtagcggtggtttttttgtttgcaagcagcagattacgcgcagaaaaaaaggatctcaagaagatc
ctttgatcttttctacggggtctgacgctcagtggaacgaaaactcacgttaagggattttggtcatgagattatcaaaaaggatcttcacc
tagatccttttaaattaaaaatgaagttttaaatcaatctaaagtatatatgagtaaacttggtctgacagttaccaatgcttaatcagtgagg
cacctatctcagcgatctgtctatttcgttcatccatagttgcctgactccccgtcgtgtagataactacgatacgggagggcttaccatct
ggccccagtgctgcaatgataccgcgagacccacgctcaccggctccagatttatcagcaataaaccagccagccggaagggccga
gcgcagaagtggtcctgcaactttatccgcctccatccagtctattaattgttgccgggaagctagagtaagtagttcgccagttaatagtt
tgcgcaacgttgttgccattgctacaggcatcgtggtgtcacgctcgtcgtttggtatggcttcattcagctccggttcccaacgatcaag
gcgagttacatgatcccccatgttgtgcaaaaaagcggttagctccttcggtcctccgatcgttgtcagaagtaagttggccgcagtgtta
tcactcatggttatggcagcactgcataattctcttactgtcatgccatccgtaagatgcttttctgtgactggtgagtactcaaccaagtcat
tctgagaatagtgtatgcggcgaccgagttgctcttgcccggcgtcaatacgggataataccgcgccacatagcagaactttaaaagtg
ctcatcattggaaaacgttcttcggggcgaaaactctcaaggatcttaccgctgttgagatccagttcgatgtaacccactcgtgcaccc
aactgatcttcagcatcttttactttcaccagcgtttctgggtgagcaaaaacaggaaggcaaaatgccgcaaaaaagggaataagggc
gacacggaaatgttgaatactcatactcttcctttttcaatattattgaagcatttatcagggttattgtctcatgagcggatacatatttgaatg
tatttagaaaaataaacaaataggggttccgcgcacatttccccgaaaagtgccacctgacgtcgacggatcgggagatctcccgatc
ccctatggtcgactctcagtacaatctgctctgatgccgcatagttaagccagtatctgctccctgcttgtgtgttggaggtcgctgagtag
tgcgcgagcaaaatttaagctacaacaaggcaaggcttgaccgacaattgcatgaagaatctgcttagg
Cas9 golden gate compatible vector (SEQ ID NO: 4):
gttaggcgttttgcgctgcttcgcgatgtacgggccagatatacgcgttgacattgattattgactagttattaatagtaatcaattacgggg
tcattagttcatagcccatatatggagttccgcgttacataacttacggtaaatggcccgcctggctgaccgcccaacgacccccgccc
attgacgtcaataatgacgtatgttcccatagtaacgccaatagggactttccattgacgtcaatgggtggagtatttacggtaaactgcc
cacttggcagtacatcaagtgtatcatatgccaagtacgccccctattgacgtcaatgacggtaaatggcccgcctggcattatgcccag
tacatgaccttatgggactttcctacttggcagtacatctacgtattagtcatcgctattaccatggtgatgcggttttggcagtacatcaatg
ggcgtggatagcggtttgactcacggggatttccaagtctccaccccattgacgtcaatgggagtttgttttggcaccaaaatcaacggg
actttccaaaatgtcgtaacaactccgccccattgacgcaaatgggcggtaggcgtgtacggtgggaggtctatataagcagagctcgt
ttagtgaaccgtcagatcgcctggagacgccatccacgctgttttgacctccatagaagacaccgggaccgatccagcctccggactc
tagaggatcgaacccttgccaccATGGACAAGAAGTACTCCATTGGGCTCGATATCGGCACAA
ACAGCGTCGGCTGGGCCGTCATTACGGACGAGTACAAGGTGCCGAGCAAAAAAT
TCAAAGTTCTGGGCAATACCGATCGCCACAGCATAAAGAAGAACCTCATTGGCG
CCCTCCTGTTCGACTCCGGGGAGACGGCCGAAGCCACGCGGCTCAAAAGAACAG
CACGGCGCAGATATACCCGCAGAAAGAATCGGATCTGCTACCTGCAGGAGATCT
TTAGTAATGAGATGGCTAAGGTGGATGACTCTTTCTTCCATAGGCTGGAGGAGTC
CTTTTTGGTGGAGGAGGATAAAAAGCACGAGCGCCACCCAATCTTTGGCAATAT
CGTGGACGAGGTGGCGTACCATGAAAAGTACCCAACCATATATCATCTGAGGAA
GAAGCTTGTAGACAGTACTGATAAGGCTGACTTGCGGTTGATCTATCTCGCGCTG
GCGCATATGATCAAATTTCGGGGACACTTCCTCATCGAGGGGGACCTGAACCCA
GACAACAGCGATGTCGACAAACTCTTTATCCAACTGGTTCAGACTTACAATCAGC
TTTTCGAAGAGAACCCGATCAACGCATCCGGAGTTGACGCCAAAGCAATCCTGA
GCGCTAGGCTGTCCAAATCCCGGCGGCTCGAAAACCTCATCGCACAGCTCCCTGG
GGAGAAGAAGAACGGCCTGTTTGGTAATCTTATCGCCCTGTCACTCGGGCTGACC
CCCAACTTTAAATCTAACTTCGACCTGGCCGAAGATGCCAAGCTTCAACTGAGCA
AAGACACCTACGATGATGATCTCGACAATCTGCTGGCCCAGATCGGCGACCAGT
ACGCAGACCTTTTTTTGGCGGCAAAGAACCTGTCAGACGCCATTCTGCTGAGTGA
TATTCTGCGAGTGAACACGGAGATCACCAAAGCTCCGCTGAGCGCTAGTATGAT
CAAGCGCTATGATGAGCACCACCAAGACTTGACTTTGCTGAAGGCCCTTGTCAGA
CAGCAACTGCCTGAGAAGTACAAGGAAATTTTCTTCGATCAGTCTAAAAATGGCT
ACGCCGGATACATTGACGGCGGAGCAAGCCAGGAGGAATTTTACAAATTTATTA
AGCCCATCTTGGAAAAAATGGACGGCACCGAGGAGCTGCTGGTAAAGCTTAACA
GAGAAGATCTGTTGCGCAAACAGCGCACTTTCGACAATGGAAGCATCCCCCACC
AGATTCACCTGGGCGAACTGCACGCTATCCTCAGGCGGCAAGAGGATTTCTACCC
CTTTTTGAAAGATAACAGGGAAAAGATTGAGAAAATCCTCACATTTCGGATACC
CTACTATGTAGGCCCCCTCGCCCGGGGAAATTCCAGATTCGCGTGGATGACTCGC
AAATCAGAAGAGACCATCACTCCCTGGAACTTCGAGGAAGTCGTGGATAAGGGG
GCCTCTGCCCAGTCCTTCATCGAAAGGATGACTAACTTTGATAAAAATCTGCCTA
ACGAAAAGGTGCTTCCTAAACACTCTCTGCTGTACGAGTACTTCACAGTTTATAA
CGAGCTCACCAAGGTCAAATACGTCACAGAAGGGATGAGAAAGCCAGCATTCCT
GTCTGGAGAGCAGAAGAAAGCTATCGTGGACCTCCTCTTCAAGACGAACCGGAA
AGTTACCGTGAAACAGCTCAAAGAAGACTATTTCAAAAAGATTGAATGTTTCGA
CTCTGTTGAAATCAGCGGAGTGGAGGATCGCTTCAACGCATCCCTGGGAACGTAT
CACGATCTCCTGAAAATCATTAAAGACAAGGACTTCCTGGACAATGAGGAGAAC
GAGGACATTCTTGAGGACATTGTCCTCACCCTTACGTTGTTTGAAGATAGGGAGA
TGATTGAAGAACGCTTGAAAACTTACGCTCATCTCTTCGACGACAAAGTCATGAA
ACAGCTCAAGAGGCGCCGATATACAGGATGGGGGCGGCTGTCAAGAAAACTGAT
CAATGGGATCCGAGACAAGCAGAGTGGAAAGACAATCCTGGATTTTCTTAAGTC
CGATGGATTTGCCAACCGGAACTTCATGCAGTTGATCCATGATGACTCTCTCACC
TTTAAGGAGGACATCCAGAAAGCACAAGTTTCTGGCCAGGGGGACAGTCTTCAC
GAGCACATCGCTAATCTTGCAGGTAGCCCAGCTATCAAAAAGGGAATACTGCAG
ACCGTTAAGGTCGTGGATGAACTCGTCAAAGTAATGGGAAGGCATAAGCCCGAG
AATATCGTTATCGAGATGGCCCGAGAGAACCAAACTACCCAGAAGGGACAGAAG
AACAGTAGGGAAAGGATGAAGAGGATTGAAGAGGGTATAAAAGAACTGGGGTC
CCAAATCCTTAAGGAACACCCAGTTGAAAACACCCAGCTTCAGAATGAGAAGCT
CTACCTGTACTACCTGCAGAACGGCAGGGACATGTACGTGGATCAGGAACTGGA
CATCAATCGGCTCTCCGACTACGACGTGGATCATATCGTGCCCCAGTCTTTTCTC
AAAGATGATTCTATTGATAATAAAGTGTTGACAAGATCCGATAAAAATAGAGGG
AAGAGTGATAACGTCCCCTCAGAAGAAGTTGTCAAGAAAATGAAAAATTATTGG
CGGCAGCTGCTGAACGCCAAACTGATCACACAACGGAAGTTCGATAATCTGACT
AAGGCTGAACGAGGTGGCCTGTCTGAGTTGGATAAAGCCGGCTTCATCAAAAGG
CAGCTTGTTGAGACACGCCAGATCACCAAGCACGTGGCCCAAATTCTCGATTCAC
GCATGAACACCAAGTACGATGAAAATGACAAACTGATTCGAGAGGTGAAAGTTA
TTACTCTGAAGTCTAAGCTGGTCTCAGATTTCAGAAAGGACTTTCAGTTTTATAA
GGTGAGAGAGATCAACAATTACCACCATGCGCATGATGCCTACCTGAATGCAGT
GGTAGGCACTGCACTTATCAAAAAATATCCCAAGCTTGAATCTGAATTTGTTTAC
GGAGACTATAAAGTGTACGATGTTAGGAAAATGATCGCAAAGTCTGAGCAGGAA
ATAGGCAAGGCCACCGCTAAGTACTTCTTTTACAGCAATATTATGAATTTTTTCA
AGACCGAGATTACACTGGCCAATGGAGAGATTCGGAAGCGACCACTTATCGAAA
CAAACGGAGAAACAGGAGAAATCGTGTGGGACAAGGGTAGGGATTTCGCGACA
GTCCGGAAGGTCCTGTCCATGCCGCAGGTGAACATCGTTAAAAAGACCGAAGTA
CAGACCGGAGGCTTCTCCAAGGAAAGTATCCTCCCGAAAAGGAACAGCGACAAG
CTGATCGCACGCAAAAAAGATTGGGACCCCAAGAAATACGGCGGATTCGATTCT
CCTACAGTCGCTTACAGTGTACTGGTTGTGGCCAAAGTGGAGAAAGGGAAGTCT
AAAAAACTCAAAAGCGTCAAGGAACTGCTGGGCATCACAATCATGGAGCGATCA
AGCTTCGAAAAAAACCCCATCGACTTTCTCGAGGCGAAAGGATATAAAGAGGTC
AAAAAAGACCTCATCATTAAGCTTCCCAAGTACTCTCTCTTTGAGCTTGAAAACG
GCCGGAAACGAATGCTCGCTAGTGCGGGCGAGCTGCAGAAAGGTAACGAGCTGG
CACTGCCCTCTAAATACGTTAATTTCTTGTATCTGGCCAGCCACTATGAAAAGCT
CAAAGGGTCTCCCGAAGATAATGAGCAGAAGCAGCTGTTCGTGGAACAACACAA
ACACTACCTTGATGAGATCATCGAGCAAATAAGCGAATTCTCCAAAAGAGTGAT
CCTCGCCGACGCTAACCTCGATAAGGTGCTTTCTGCTTACAATAAGCACAGGGAT
AAGCCCATCAGGGAGCAGGCAGAAAACATTATCCACTTGTTTACTCTGACCAACT
TGGGCGCGCCTGCAGCCTTCAAGTACTTCGACACCACCATAGACAGAAAGCGGT
ACACCTCTACAAAGGAGGTCCTGGACGCCACACTGATTCATCAGTCAATTACGG
GGCTCTATGAAACAAGAATCGACCTCTCTCAGCTCGGTGGAGACAGCAGGGCTG
ACCCCAAGAAGAAGAGGAAGGTGagtggtggaggaagttgaagagctatgtttagatatccaaaccaggctctt
cttagaagaattcgatccctaccggttagtaatgagtttaaacgggggaggctaactgaaacacggaaggagacaataccggaagga
acccgcgctatgacggcaataaaaagacagaataaaacgcacgggtgttgggtcgtttgttcataaacgcggggttcggtcccaggg
ctggcactctgtcgataccccaccgagaccccattggggccaatacgcccgcgtttcttccttttccccaccccaccccccaagttcgg
gtgaaggcccagggctcgcagccaacgtcggggcggcaggccctccatagtcggtcgttcggctgcggcgagcggtatcagctca
ctcaaaggcggtaatacggttatccacagaatcaggggataacgcaggaaagaacatgtgagcaaaaggccagcaaaaggccagg
aaccgtaaaaaggccgcgttgctggcgtttttccataggctccgcccccctgacgagcatcacaaaaatcgacgctcaagtcagaggt
ggcgaaacccgacaggactataaagataccaggcgtttccccctggaagctccctcgtgcgctctcctgttccgaccctgccgcttac
cggatacctgtccgcctttctcccttcgggaagcgtggcgctttctcatagctcacgctgtaggtatctcagttcggtgtaggtcgttcgct
ccaagctgggctgtgtgcacgaaccccccgttcagcccgaccgctgcgccttatccggtaactatcgtcttgagtccaacccggtaag
acacgacttatcgccactggcagcagccactggtaacaggattagcagagcgaggtatgtaggcggtgctacagagttcttgaagtg
gtggcctaactacggctacactagaaggacagtatttggtatctgcgctctgctgaagccagttaccttcggaaaaagagttggtagctc
ttgatccggcaaacaaaccaccgctggtagcggtggtttttttgtttgcaagcagcagattacgcgcagaaaaaaaggatctcaagaag
atcctttgatcttttctacggggtctgacgctcagtggaacgaaaactcacgttaagggattttggtcatgagattatcaaaaaggatcttc
acctagatccttttaaattaaaaatgaagttttaaatcaatctaaagtatatatgagtaaacttggtctgacagttaccaatgcttaatcagtga
ggcacctatctcagcgatctgtctatttcgttcatccatagttgcctgactccccgtcgtgtagataactacgatacgggagggcttaccat
ctggccccagtgctgcaatgataccgcgagacccacgctcaccggctccagatttatcagcaataaaccagccagccggaagggcc
gagcgcagaagtggtcctgcaactttatccgcctccatccagtctattaattgttgccgggaagctagagtaagtagttcgccagttaata
gtttgcgcaacgttgttgccattgctacaggcatcgtggtgtcacgctcgtcgtttggtatggcttcattcagctccggttcccaacgatca
aggcgagttacatgatcccccatgttgtgcaaaaaagcggttagctccttcggtcctccgatcgttgtcagaagtaagttggccgcagtg
ttatcactcatggttatggcagcactgcataattctcttactgtcatgccatccgtaagatgcttttctgtgactggtgagtactcaaccaagt
cattctgagaatagtgtatgcggcgaccgagttgctcttgcccggcgtcaatacgggataataccgcgccacatagcagaactttaaaa
gtgctcatcattggaaaacgttcttcggggcgaaaactctcaaggatcttaccgctgttgagatccagttcgatgtaacccactcgtgcac
ccaactgatcttcagcatcttttactttcaccagcgtttctgggtgagcaaaaacaggaaggcaaaatgccgcaaaaaagggaataagg
gcgacacggaaatgttgaatactcatactcttcctttttcaatattattgaagcatttatcagggttattgtctcatgagcggatacatatttga
atgtatttagaaaaataaacaaataggggttccgcgcacatttccccgaaaagtgccacctgacgtcgacggatcgggagatctcccg
atcccctatggtcgactctcagtacaatctgctctgatgccgcatagttaagccagtatctgctccctgcttgtgtgttggaggtcgctgag
tagtgcgcgagcaaaatttaagctacaacaaggcaaggcttgaccgacaattgcatgaagaatctgcttagg
gRNA cloning vector pSB700-SAM (SEQ ID NO: 5):
tggaagggctaattcactcccaaagaagacaagatatccttgatctgtggatctaccacacacaaggctacttccctgattagcagaact
acacaccagggccaggggtcagatatccactgacctttggatggtgctacaagctagtaccagttgagccagataaggtagaagagg
ccaataaaggagagaacaccagcttgttacaccctgtgagcctgcatgggatggatgacccggagagagaagtgttagagtggaggt
ttgacagccgcctagcatttcatcacgtggcccgagagctgcatccggagtacttcaagaactgctgatatcgagcttgctacaaggga
ctttccgctggggactttccagggaggcgtggcctgggcgggactggggagtggcgagccctcagatcctgcatataagcagctgct
ttttgcctgtactgggtctctctggttagaccagatctgagcctgggagctctctggctaactagggaacccactgcttaagcctcaataa
agcttgccttgagtgcttcaagtagtgtgtgcccgtctgttgtgtgactctggtaactagagatccctcagacccttttagtcagtgtggaa
aatctctagcagtggcgcccgaacagggacttgaaagcgaaagggaaaccagaggagctctctcgacgcaggactcggcttgctga
agcgcgcacggcaagaggcgaggggcggcgactggtgagtacgccaaaaattttgactagcggaggctagaaggagagagatgg
gtgcgagagcgtcagtattaagcgggggagaattagatcgcgatgggaaaaaattcggttaaggccagggggaaagaaaaaatata
aattaaaacatatagtatgggcaagcagggagctagaacgattcgcagttaatcctggcctgttagaaacatcagaaggctgtagacaa
atactgggacagctacaaccatcccttcagacaggatcagaagaacttagatcattatataatacagtagcaaccctctattgtgtgcatc
aaaggatagagataaaagacaccaaggaagctttagacaagatagaggaagagcaaaacaaaagtaagaccaccgcacagcaag
cggccggccgctgatcttcagacctggaggaggagatatgagggacaattggagaagtgaattatataaatataaagtagtaaaaatt
gaaccattaggagtagcacccaccaaggcaaagagaagagtggtgcagagagaaaaaagagcagtgggaataggagctttgttcct
tgggttcttgggagcagcaggaagcactatgggcgcagcgtcaatgacgctgacggtacaggccagacaattattgtctggtatagtg
cagcagcagaacaatttgctgagggctattgaggcgcaacagcatctgttgcaactcacagtctggggcatcaagcagctccaggca
agaatcctggctgtggaaagatacctaaaggatcaacagctcctggggatttggggttgctctggaaaactcatttgcaccactgctgtg
ccttggaatgctagttggagtaataaatctctggaacagatttggaatcacacgacctggatggagtgggacagagaaattaacaattac
acaagcttaatacactccttaattgaagaatcgcaaaaccagcaagaaaagaatgaacaagaattattggaattagataaatgggcaag
tttgtggaattggtttaacataacaaattggctgtggtatataaaattattcataatgatagtaggaggcttggtaggtttaagaatagtttttg
ctgtactttctatagtgaatagagttaggcagggatattcaccattatcgtttcagacccacctcccaaccccgaggggacccgacaggc
ccgaaggaatagaagaagaaggtggagagagagacagagacagatccattcgattagtgaacggatctcgacggtatcgcctttaaa
agaaaaggggggattggggggtacagtgcaggggaaagaatagtagacataatagcaacagacatacaaactaaagaattacaaaa
acaaattacaaaaattcaaaattttcgggtttattacagggacagcagagatccagtttatcattagtgaacggatctcgacggtatcgatc
acgagactagcctcgagcggccgcccccttcaccgagggcctatttcccatgattccttcatatttgcatatacgatacaaggctgttag
agagataattggaattaatttgactgtaaacacaaagatattagtacaaaatacgtgacgtagaaagtaataatttcttgggtagtttgcagt
tttaaaattatgttttaaaatggactatcatatgcttaccgtaacttgaaagtatttcgatttcttggctttatatatcttgtggaaaggacgaaa
caccggagacgattaatgcgtctcgGTTTTAGAGCTAGGCCAACATGAGGATCACCCATGTCTG
CAGGGCCTAGCAAGTTAAAATAAGGCTAGTCCGTTATCAACTTGGCCAACATGA
GGATCACCCATGTCTGCAGGGCCAAGTGGCACCGAGTCGGTGCTTTTTttgaattctcga
cctcgagacaaatggcagtattcgtcattagttcatagcccatatatggagttccgcgttacataacttacggtaaatggcccgcctggct
gaccgcccaacgacccccgcccattgacgtcaataatgacgtatgttcccatagtaacgccaatagggactttccattgacgtcaatgg
gtggagtatttacggtaaactgcccacttggcagtacatcaagtgtatcatatgccaagtacgccccctattgacgtcaatgacggtaaat
ggcccgcctggcattatgcccagtacatgaccttatgggactttcctacttggcagtacatctacgtattagtcatcgctattaccatggtc
gaggtgagccccacgttctgcttcactctccccatctcccccccctccccacccccaattttgtatttatttattttttaattattttgtgcagcg
atgggggcgggggggggggggcgcgcgccaggcggggcggggcggggcgaggggcggggcggggcgaggcggagaggt
gcggcggcagccaatcagagcggcgcgctccgaaagtttccttttatggcgaggcggcggcggcggcggccctataaaaagcgaa
gcgcgcggcgggcggggagtcgctgcgacgctgccttcgccccgtgccccgctccgccgccgcctcgcgccgcccgccccggct
ctgactgaccgcgttactcccacaggtgagcgggcgggacggcccttctcctccgggctgtaattagcgcttggtttaatgacggcttg
tttcttttctgtggctgcgtgaaagccttgaggggctccgggagggccctttgtgcggggggagcggctcggggggtgcgtgcgtgtg
tgtgtgcgtggggagcgccgcgtgcggctccgcgctgcccggcggctgtgagcgctgcgggcgcggcgcggggctttgtgcgctc
cgcagtgtgcgcgaggggagcgcggccgggggcggtgccccgcggtgcggggggggctgcgaggggaacaaaggctgcgtg
cggggtgtgtgcgtgggggggtgagcagggggtgtgggcgcgtcggtcgggctgcaaccccccctgcacccccctccccgagttg
ctgagcacggcccggcttcgggtgcggggctccgtacggggcgtggcgcggggctcgccgtgccgggcggggggtggcggca
ggtgggggtgccgggcggggcggggccgcctcgggccggggagggctcgggggaggggcgcggcggcccccggagcgccg
gcggctgtcgaggcgcggcgagccgcagccattgccttttatggtaatcgtgcgagagggcgcagggacttcctttgtcccaaatctg
tgcggagccgaaatctgggaggcgccgccgcaccccctctagcgggcgcggggcgaagcggtgcggcgccggcaggaaggaa
atgggcggggagggccttcgtgcgtcgccgcgccgccgtccccttctccctctccagcctcggggctgtccgcggggggacggctg
ccttcgggggggacggggcagggcggggttcggcttctggcgtgtgaccggcggctctagagcctctgctaaccatgttcatgcctt
cttctttttcctacagctcctgggcaacgtgctggttattgtggccaccatggtgagcaagggcgaggagctgttcaccggggtggtgc
ccatcctggtcgagctggacggcgacgtaaacggccacaagttcagcgtgtccggcgagggcgagggcgatgccacctacggcaa
gctgaccctgaagttcatctgcaccaccggcaagctgcccgtgccctggcccaccctcgtgaccaccctgacctacggcgtgcagtg
cttcagccgctaccccgaccacatgaagcagcacgacttcttcaagtccgccatgcccgaaggctacgtccaggagcgcaccatcttc
ttcaaggacgacggcaactacaagacccgcgccgaggtgaagttcgagggcgacaccctggtgaaccgcatcgagctgaagggc
atcgacttcaaggaggacggcaacatcctggggcacaagctggagtacaactacaacagccacaacgtctatatcatggccgacaa
gcagaagaacggcatcaaggtgaacttcaagatccgccacaacatcgaggacggcagcgtgcagctcgccgaccactaccagcag
aacacccccatcggcgacggccccgtgctgctgcccgacaaccactacctgagcacccagtccgccctgagcaaagaccccaacg
agaagcgcgatcacatggtcctgctggagttcgtgaccgccgccgggatcactctcggcatggacgagctgtacaagtaaagcgtct
ggaacaatcaacctctggattacaaaatttgtgaaagattgactggtattcttaactatgttgctccttttacgctatgtggatacgctgcttta
atgcctttgtatcatgcgcttcccgtatggctttcattttctcctccttgtataaatcctggttgctgtctctttatgaggagttgtggcccg
ttgtcaggcaacgtggcgtggtgtgcactgtgtttgctgacgcaacccccactggttggggcattgccaccacctgtcagctcctttccg
ggactttcgctttccccctccctattgccacggcggaactcatcgccgcctgccttgcccgctgctggacaggggctcggctgttgggc
actgacaattccgtggtgttgtcggggaagctgacgtcctttccatggctgctcgcctgtgttgccacctggattctgcgcgggacgtcc
ttctgctacgtcccttcggccctcaatccagcggaccttccttcccgcggcctgctgccggctctgcggcctcttccgcgtcttcgccttc
gccctcagacgagtcggatctccctttgggccgcctccccgcctggaattaattctgcagtcgagacctagaaaaacatggagcaatc
acaagtagcaatacagcagctaccaatgctgattgtgcctggctagaagcacaagaggaggaggaggtgggttttccagtcacacct
caggtacctttaagaccaatgacttacaaggcagctgtagatcttagccactttttaaaagaaaagaggggactggaagggctaattca
ctcccaacgaagacaagatatccttgatctgtggatctaccacacacaaggctacttccctgattagcagaactacacaccagggccag
gggtcagatatccactgacctttggatggtgctacaagctagtaccagttgagccagataaggtagaagaggccaataaaggagaga
acaccagcttgttacaccctgtgagcctgcatgggatggatgacccggagagagaagtgttagagtggaggtttgacagccgcctag
catttcatcacgtggcccgagagctgcatccggagtacttcaagaactgctgatatcgagcttgctacaagggactttccgctggggac
tttccagggaggcgtggcctgggcgggactggggagtggcgagccctcagatcctgcatataagcagctgctttttgcctgtactggg
tctctctggttagaccagatctgagcctgggagctctctggctaactagggaacccactgcttaagcctcaataaagcttgccttgagtg
cttcaagtagtgtgtgcccgtctgttgtgtgactctggtaactagagatccctcagacccttttagtcagtgtggaaaatctctagcagtagt
agttcatgtcatcttattattcagtatttataacttgcaaagaaatgaatatcagagagtgagaggccttgacattgctagcgttttaccgtcg
acctctagctagagcttggcgtaatcatggtcatagctgtttcctgtgtgaaattgttatccgctcacaattccacacaacatacgagccgg
aagcataaagtgtaaagcctggggtgcctaatgagtgagctaactcacattaattgcgttgcgctcactgcccgctttccagtcgggaaa
cctgtcgtgccagctgcattaatgaatcggccaacgcgcggggagaggcggtttgcgtattgggcgctcttccgcttcctcgctcactg
actcgctgcgctcggtcgttcggctgcggcgagcggtatcagctcactcaaaggcggtaatacggttatccacagaatcaggggataa
cgcaggaaagaacatgtgagcaaaaggccagcaaaaggccaggaaccgtaaaaaggccgcgttgctggcgtttttccataggctcc
gcccccctgacgagcatcacaaaaatcgacgctcaagtcagaggtggcgaaacccgacaggactataaagataccaggcgtttccc
cctggaagctccctcgtgcgctctcctgttccgaccctgccgcttaccggatacctgtccgcctttctcccttcgggaagcgtggcgcttt
ctcatagctcacgctgtaggtatctcagttcggtgtaggtcgttcgctccaagctgggctgtgtgcacgaaccccccgttcagcccgac
cgctgcgccttatccggtaactatcgtcttgagtccaacccggtaagacacgacttatcgccactggcagcagccactggtaacaggat
tagcagagcgaggtatgtaggcggtgctacagagttcttgaagtggtggcctaactacggctacactagaagaacagtatttggtatct
gcgctctgctgaagccagttaccttcggaaaaagagttggtagctcttgatccggcaaacaaaccaccgctggtagcggtggtttttttg
tttgcaagcagcagattacgcgcagaaaaaaaggatctcaagaagatcctttgatcttttctacggggtctgacgctcagtggaacgaa
aactcacgttaagggattttggtcatgagattatcaaaaaggatcttcacctagatccttttaaattaaaaatgaagttttaaatcaatctaaa
gtatatatgagtaaacttggtctgacagttaccaatgcttaatcagtgaggcacctatctcagcgatctgtctatttcgttcatccatagttgc
ctgactccccgtcgtgtagataactacgatacgggagggcttaccatctggccccagtgctgcaatgataccgcgagacccacgctca
ccggctccagatttatcagcaataaaccagccagccggaagggccgagcgcagaagtggtcctgcaactttatccgcctccatccagt
ctattaattgttgccgggaagctagagtaagtagttcgccagttaatagtttgcgcaacgttgttgccattgctacaggcatcgtggtgtca
cgctcgtcgtttggtatggcttcattcagctccggttcccaacgatcaaggcgagttacatgatcccccatgttgtgcaaaaaagcggtta
gctccttcggtcctccgatcgttgtcagaagtaagttggccgcagtgttatcactcatggttatggcagcactgcataattctcttactgtca
tgccatccgtaagatgcttttctgtgactggtgagtactcaaccaagtcattctgagaatagtgtatgcggcgaccgagttgctcttgccc
ggcgtcaatacgggataataccgcgccacatagcagaactttaaaagtgctcatcattggaaaacgttcttcggggcgaaaactctcaa
ggatcttaccgctgttgagatccagttcgatgtaacccactcgtgcacccaactgatcttcagcatcttttactttcaccagcgtttctgggt
gagcaaaaacaggaaggcaaaatgccgcaaaaaagggaataagggcgacacggaaatgttgaatactcatactcttcctttttcaatat
tattgaagcatttatcagggttattgtctcatgagcggatacatatttgaatgtatttagaaaaataaacaaataggggttccgcgcacattt
ccccgaaaagtgccacctgacgtcgacggatcgggagatcaacttgtttattgcagcttataatggttacaaataaagcaatagcatcac
aaatttcacaaataaagcatttttttcactgcattctagttgtggtttgtccaaactcatcaatgtatcttatcatgtctggatcaactggataac
tcaagctaaccaaaatcatcccaaacttcccaccccataccctattaccactgccaattacctgtggtttcatttactctaaacctgtgattc
ctctgaattatiticattttaaagaaattgtatttgttaaatatgtactacaaacttagtagt

Spacer sequences for endogenous gene targeting

| (SEQ ID NO: 6) | | |
|---|---|---|
| TTN | −169 | CCTTGGTGAAGTCTCCTTTG |
| (SEQ ID NO: 7) | | |
| MIAT | −219 | ATGCGGGAGGCTGAGCGCAC |

Protein sequence for DNA binding domains fused to Cas9 proteins:

Ctb peptide sequence:
GGSGSSSTSTTAKRKKRKL (SEQ ID NO:8)
sfGFP + 15 (SEQ ID NO: 9):
GGASKGERLFTGVVPILVELDGDVNGHKFSVRGEGEGDATRGKLTLKFIC
TTGKLPVPWPTLVTTLTYGVQCFSRYPKHMKRHDFFKSAMPEGYVQERTI
SFKKDGTYKTRAEVKFEGRTLVNRIELKGRDFKEKGNILGHKLEYNFNSH
NVYITADKRKNGIKANFKIRHNVKDGSVQLADHYQQNTPIGRGPVLLPRN
HYLSTRSALSKDPKEKRDHMVLLEFVTAAGITHGMDELYK
sssIM methyltransferase, with methyltransferase
point mutation (A186E)(SEQ ID NO: 10):
MSKVENKTKKLRVFEAFAGIGAQRKALEKVRKDEYEIVGLAEWYVPAIVM
YQAIHNNFHTKLEYKSVSREEMIDYLENKTLSWNSKNPVSNGYWKRKKDD
ELKIIYNAIKLSEKEGNIFDIRDLYKRTLKNIDLLTYSFPCQDLSQQGIQ
KGMKRGSGTRSGLLWEIERALDSTEKNDLPKYLLMANVGALLHKKNEEEL
NQWKQKLESLGYQNSIEVLNAADFGSSQARRRVFMISTLNEFVELPKGDK
KPKSIKKVLNKIVSEKDILNNLLKYNLTEFKKTKSNINKASLIGYSKFNS
EGYVYDPEFTGPTLTASGANSRIKIKDGSNIRKMNSDETFLYIGFDSQDG
KRVNEIEFLTENQKIFVCGNSISVEVLEAIIDKIGG
sso7d (SEQ ID NO: 11):
MATVKFKYKGEEKEVDISKIKKVWRVGKMISFTYDEGGGKTGRGAVSEKD
APKELLQMLEKQKK

### EXAMPLE II

### Mutant Cas9 with Reduced Electrostatic Repulsion Provides Increased Activation

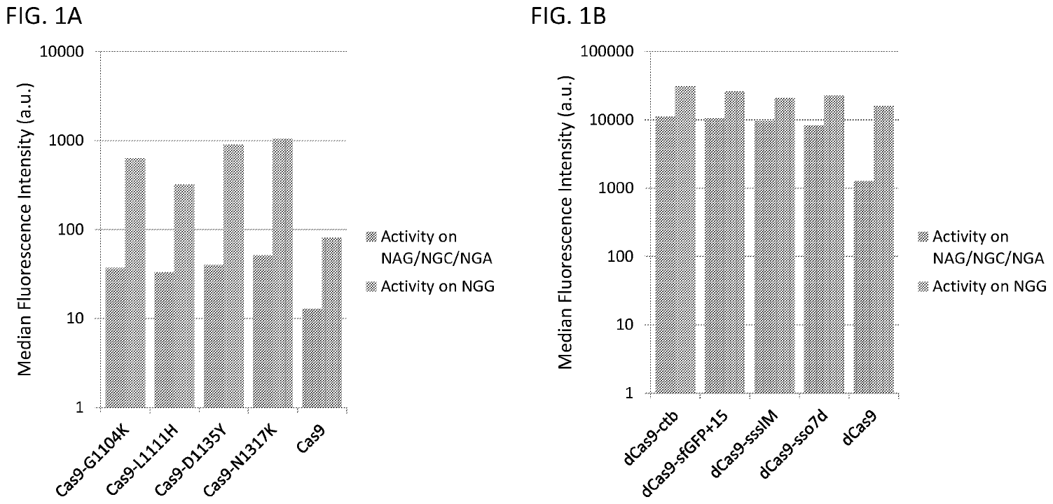

The following examples are directed to SP-Cas9 unless otherwise indicated. The disclosure provides that reducing electrostatic repulsion between Cas9 and DNA improves the ability for Cas9 to bind DNA. A series of Cas9 point mutants (G1104K, L1111H, D1135Y and N1317K) were generated which alter a negatively charged or neutral residue within the Cas9 protein to a neutral or positively charged residue. Each of these mutants was then directed to a transcriptional reporter containing a canonical NGG PAM upstream of a fluorescent reporter in conjunction with a gRNA capable of recruiting the potent MS2-p65-hsf1 activator. A marked increase in activation was observed for all of the Cas9 point mutants over the wild-type Cas9 scaffold (see FIG. 1A). Consistent with the increased activation on an NGG PAM, binding to a non-canonical PAM (NAG, NGA or NGC) is improved by the various point mutants as tested using a similar reporter assay. As with the NGG PAM, when cells were transfected with a mixture of reporters containing non-canonical PAMs the various mutant Cas9 proteins each showed an increased ability to bind and activate the reporter (see FIG. 1A). The disclosure provides for methods of introducing charge altering mutations to the Cas9 scaffold to improve improved DNA binding.

### EXAMPLE III

### dCas9-DNA Binding Peptide, Protein or Domain Fusions Improve Binding

The disclosure provides for methods of improving Cas9 DNA targeting using a fusion of Cas9 to proteins with DNA binding capacity or small positively charged peptides. In cases where the chosen DNA binding protein contained enzymatic activity outside of the ability to bind DNA (such as sssIM), residues involved in enzymatic activity but not DNA binding were mutated to render the protein competent for DNA binding but not other activities before fusing the given protein to dCas9. The various dCas9 fusions were then directed to a transcriptional reporter containing a canonical NGG PAM upstream of a fluorescent reporter. An increase in DNA binding was observed for all of the dCas9 fusions over wild-type dCas9 protein (see FIG. 1B). The level of activation on non-canonical PAMs (NAG, NGA, or NGC) was characterized to demonstrate a similar increase in DNA binding for all Cas9 fusions (see FIG. 1B). The disclosure provides for methods of further enhance Cas9 binding to target nucleic acids using a fusion of a protein with DNA binding capacity to Cas9.

### EXAMPLE IV

### Gene Activation by Mutant Cas9 or dCas9-DNA Binding Protein Fusions

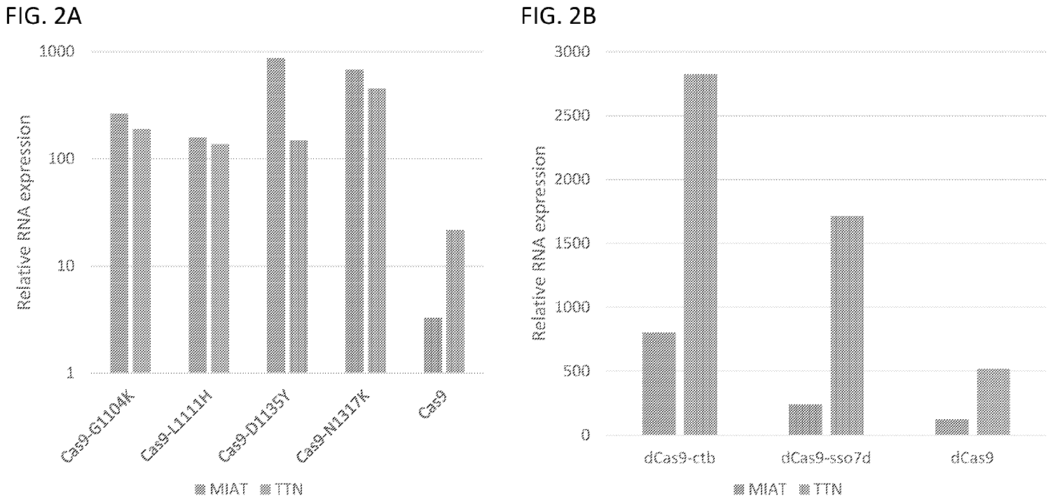

The disclosure provides methods of increased targeting to a set of native loci within cells, such as HEK293T cells, for gene activation using a Cas9 mutant or dCas9-DNA binding protein fusion and transcriptional activators. When directed to the promoters of TTN and MIAT with gRNAs capable of recruiting the MS2-p65-hsf1 activator, a marked increase in gene activation was observed for all of the Cas9 variants over their Cas9 or dCas9 controls, respectively (see FIGS. 2A and 2B).

### EXAMPLE V

### Gene Repression by Mutant Cas9 or dCas9-DNA Binding Protein Fusions

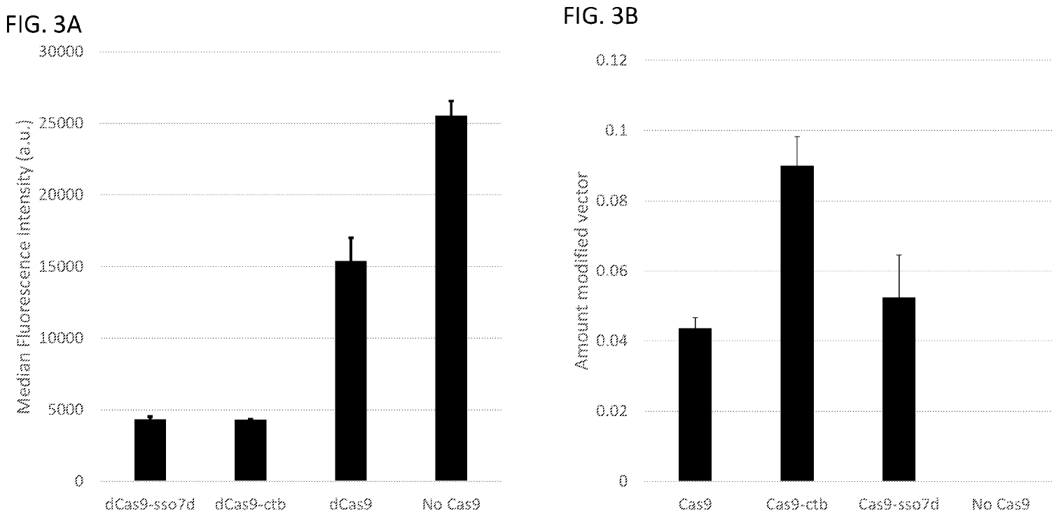

The disclosure provides methods of increased targeting to a set of native loci within cells for gene repression or genome modification using a Cas9 mutant or dCas9-DNA binding protein fusion and transcriptional repressors. As an example, the disclosure provides methods of increased targeting to a plasmid reporter for gene repression or genome modification using a Cas9 mutant or dCas9-DNA binding protein fusion and transcriptional repressors. As compared to wild-type dCas9, dCas9 fusions showed a marked improvement in repression as determined by a fluorescent reporter assay (see FIG. 3A). When DNA fusions were attached to nuclease competent Cas9 and targeted to a reporter to assay for target site deletion, an increase in the ratio of modified reporter plasmids was observed for dCas9 fusions (see FIG. 3B).

### EXAMPLE VI

### dCas9-DNA Binding Protein Fusions Enhance Activity Across Various Cas Proteins

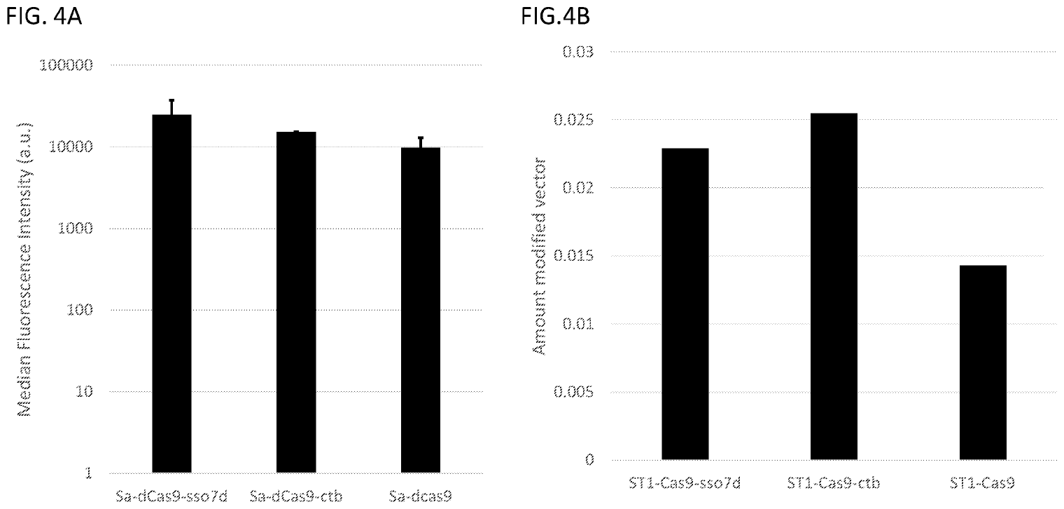

Two Cas9 orthologues Staphylococcus aureus (SA)-Cas9 and Streptococcus thermophilus (ST1)-Cas9 were fused to DNA binding enhancing proteins. Nuclease null SA-Cas9 when fused to DNA binding enhancing proteins showed a marked increase in gene activation as compared to the non-fused nuclease null SA-Cas9 control (see FIG. 4A). When nuclease competent ST1-Cas9 was fused to the DNA binding enhancing proteins, the fusion proteins were better able to modify a deletion reporter as compare to the non-fused ST1 control (see FIG. 4B).

### EXAMPLE VII

### Fusion of DNA Binding Peptides/Proteins to Cas9 Enhance Activity at Endogenous Chromosomal Loci

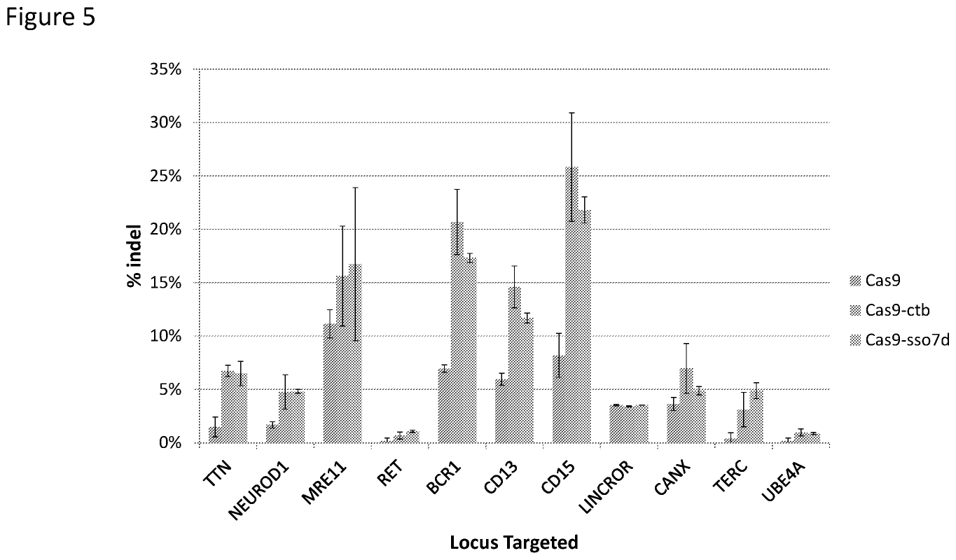

To determine if the fusion of DNA binding proteins to Cas9 would enhance Cas9 cutting activity across a range of target sites, mammalian HEK293T cells were transfected with guides targeting a series of endogenous sites along with either wild-type Cas9 or a Cas9 fusion construct. Across the genomic sites tested, the exemplary Cas9 fusion proteins (Cas9-ctb and Cas9-sso7d) showed improved activity compared with Cas9 with up to a 12-fold increase in the rate of detected mutations (indels) as determined by next-generation sequencing. See FIG. 5.

For experiments involving next generation sequencing, 293T cells within a 24-well plate were transfected with either Cas9-ctb or Cas9-sso7d (50 ng), a plasmid conferring puromycin resistance (50 ng) and with a mixture of guide RNAs designed to direct Cas9 to each of the noted target genes (100 ng of total gRNA plasmid added). 24 hours post transfection the cells were treated with 3 ug/ml of puromycin, and 48 hours after the addition of puromycin, DNA was harvested from the cells using QuickExtract DNA Extraction Solution (Epicenter) according to the manufacturer's protocol. A multiplex PCR reaction was then performed to selectively amplify each of the target loci within a single PCR reaction. A second round of PCR was then performed to index the samples for subsequent sequencing on a Miseq DNA sequencer (Illumina). Custom scripts were used to map the resulting sequencing data back to the reference sequence and to determine the fraction of mapped reads that were mutant.

The following guide RNA sequences were used in the multiplex cutting experiment:

| | |
|---|---|
| TTN: | CCTTGGTGAAGTCTCCTTTG |
| NeuroD1: | TAGAGGGGCCGACGGAGATT |
| MRE11: | AGAAAGGAAGAGTGGGGAA |
| RET: | ACACTTCCACTGTAGTCAG |
| BCR1: | TAACTCCTTGAGTGGGGCGC |
| CD13: | AAGAGAGACAGTACATGCCC |
| CD15: | ACGTGGATGAAGGCGCCGCG |
| LincROR: | CCAGGAAAAGGACTTTCACA |
| CANX: | GCGCCGCAGTAAAGAGAGAGG |
| TERC: | GTCTAACCCTAACTGAGAA |
| UBE4A: | GCGCTTGTGCGGAGCCGGAGG |

The following primers were used to amplify endogenous loci in multiplex fashion:

CTTTCCCTACACGACGCTCTTCCGATCT NNNNNN
GCGGGGACTAGACCAGAAGG
CTTTCCCTACACGACGCTCTTCCGATCT NNNNNN
TCAGGTTTGGGGCTCTTTTG
CTTTCCCTACACGACGCTCTTCCGATCT NNNNNN
GATCCGGTTAGGGAGGTTGG
CTTTCCCTACACGACGCTCTTCCGATCT NNNNNN
TCTGTATCCTTAATGGTGTTCTCTCTC
CTTTCCCTACACGACGCTCTTCCGATCT NNNNN
GCACCCAGAATGAGGTGGTC
CTTTCCCTACACGACGCTCTTCCGATCT NNNNN
TCCACACTCTGAGGCGGAAC
CTTTCCCTACACGACGCTCTTCCGATCT NNNNNN
GGAGCTCCAGATGGCTAAGG
CTTTCCCTACACGACGCTCTTCCGATCT NNNNN
ATCTTTGAGGGCCTGGGTTG
CTTTCCCTACACGACGCTCTTCCGATCT NNNNN
CAGTGGCCCGCTACAAGTTC
CTTTCCCTACACGACGCTCTTCCGATCT NNNNN
TGTTCTCTGGTGGGCAGGAG
CTTTCCCTACACGACGCTCTTCCGATCT NNNNN
CGGCTGTGGCTACTCAGG
CTTTCCCTACACGACGCTCTTCCGATCT NNNNN
AGCCGCGAGAGTCAGCTTG
CTTTCCCTACACGACGCTCTTCCGATCT NNNNN
CTGTGCTGAGTCGGAAGTGG
GGAGTTCAGACGTGTGCTCTTCCGATCT GGTCTGTGGATTCGGTCCTC
GGAGTTCAGACGTGTGCTCTTCCGATCT GGAAAGCATGATGGGAGAGG
GGAGTTCAGACGTGTGCTCTTCCGATCT TTGTCCTGACACTGGCATCC
GGAGTTCAGACGTGTGCTCTTCCGATCT TCCTGGCATTGACATTCCAC
GGAGTTCAGACGTGTGCTCTTCCGATCT CATTTCCCAAATGCGCTCTC
GGAGTTCAGACGTGTGCTCTTCCGATCT AGGCCCCTGAAAGCTGCTAC
GGAGTTCAGACGTGTGCTCTTCCGATCT ACCTTCCAGCAGGTCTGTCG
GGAGTTCAGACGTGTGCTCTTCCGATCT CCATGCACCTCCGTACCTTC
GGAGTTCAGACGTGTGCTCTTCCGATCT GGTTGCGGTCGAGGAAAAG
GGAGTTCAGACGTGTGCTCTTCCGATCT TATGCCCAGATGGCCTGAAG
GGAGTTCAGACGTGTGCTCTTCCGATCT TCTCCCCTCTCACCTCTAGCC
GGAGTTCAGACGTGTGCTCTTCCGATCT TGCTCTAGAATGAACGGTGGA
AG
GGAGTTCAGACGTGTGCTCTTCCGATCT CCAAATCAGACAGGGTCGAAG

1. A mutant Cas9 protein having target nucleic acid binding activity in the absence of an adjacent functional protospacer adjacent motif.

2. The mutant Cas9 protein of claim 1 including one or more amino acid mutations selected from the group consisting of a negatively charged amino acid to a neutral charged amino acid, a negatively charged amino acid to a positively charged amino acid and a neutral charged amino acid to a positively charged amino acid.

3. The mutant Cas9 protein of claim 1 including one or more amino acid mutations that result in the mutant Cas9 protein having a lower electrostatic repulsion to DNA compared to wild type or unmutated Cas protein.

4. The mutant Cas9 protein of claim 1 including one or more mutations selected from the group consisting of G1104K, L1111H, D1135Y and N1317K.

5. The mutant Cas9 protein of claim 1 having nuclease activity.

6. The mutant Cas9 protein of claim 1 wherein the mutant Cas9 protein is a nickase.

7. The mutant Cas9 protein wherein the mutant Cas9 protein is nuclease null.

8. The mutant Cas9 protein of claim 1 having a transcriptional regulator attached thereto.

9. A mutant Cas9 protein having increased target nucleic acid binding activity in the presence of an adjacent functional protospacer adjacent motif.

10. The mutant Cas9 protein of claim 1 including one or more amino acid mutations selected from the group consisting of a negatively charged amino acid to a neutral charged amino acid, a negatively charged amino acid to a positively charged amino acid and a neutral charged amino acid to a positively charged amino acid.

11. The mutant Cas9 protein of claim 1 including one or more amino acid mutations that result in the mutant Cas9 protein having a lower electrostatic repulsion to DNA compared to wild type or unmutated Cas protein.

12. A mutant Cas9 protein bound to a target nucleic acid, wherein the target nucleic acid lacks an adjacent functional protospacer adjacent motif.

13. A mutant Cas9 protein including one or more mutations selected from the group consisting of G1104K, L1111H, D1135Y and N1317K.

14. A method of making a mutant Cas9 protein comprising
expressing a nucleic acid sequence encoding a Cas9 protein including one or more mutations selected from the group consisting of a negatively charged amino acid to a neutral charged amino acid, a negatively charged amino acid to a positively charged amino acid and a neutral charged amino acid to a positively charged amino acid.

15. The method of claim 14 wherein the one or more mutations are selected from the group consisting of G1104K, L1111H, D1135Y and N1317K.

16. A method of altering a target nucleic acid in a cell comprising
providing to the cell a mutant Cas9 protein including one or more mutations selected from the group consisting of a negatively charged amino acid to a neutral charged amino acid, a negatively charged amino acid to a positively charged amino acid and a neutral charged amino acid to a positively charged amino acid,
providing to the cell a guide RNA including a spacer sequence complementary to a target nucleic acid, wherein the guide RNA and the mutant Cas9 protein form a co-localization complex with the target nucleic acid, and
the target nucleic acid is altered.

17. The method of claim 16 wherein the one or more mutations are selected from the group consisting of G1104K, L1111H, D1135Y and N1317K.

18. The method of claim 16 wherein the mutant Cas9 protein is an enzymatically active Cas9 and the target nucleic acid is cleaved by the mutant Cas9 protein.

19. The method of claim 16 wherein the mutant Cas9 protein is a nickase and one strand of the target nucleic acid is cleaved by the mutant Cas9 protein.

20. The method of claim 16 wherein the mutant Cas9 protein is a nuclease null Cas9 and wherein a transcriptional regulator is attached to either the mutant Cas9 protein or the guide RNA and the target nucleic acid is regulated.

21. The method of claim 16 wherein the mutant Cas9 protein is provided to the cell by introducing into the cell a first foreign nucleic acid encoding the mutant Cas9 protein and wherein the guide RNA is provided to the cell by introducing into the cell a second foreign nucleic acid encoding the guide RNA, and
wherein the guide RNA and the mutant Cas9 protein are expressed.

22. The method of claim 16 wherein the cell is in vitro, in vivo or ex vivo.

23. The method of claim 16 wherein the cell is a eukaryotic cell or prokaryotic cell.

24. The method of claim 16 wherein the cell is a bacteria cell, a yeast cell, a fungal cell, a mammalian cell, a plant cell or an animal cell.

25. The method of claim 16 wherein the target nucleic acid is genomic DNA, mitochondrial DNA, plastid DNA, viral DNA, or exogenous DNA.

26. A cell comprising
a mutant Cas9 protein including one or more mutations selected from the group consisting of a negatively charged amino acid to a neutral charged amino acid, a negatively charged amino acid to a positively charged amino acid and a neutral charged amino acid to a positively charged amino acid, and
a guide RNA and wherein the guide RNA and the Cas9 protein are members of a co-localization complex for the target nucleic acid.

27. The method of claim 26 wherein the one or more mutations are selected from the group consisting of G1104K, L1111H, D1135Y and N1317K.

28. The method of claim 26 wherein the cell is a eukaryotic cell or prokaryotic cell.

29. The method of claim 26 wherein the cell is a bacteria cell, a yeast cell, a fungal cell, a mammalian cell, a plant cell or an animal cell.

30. A cell comprising
a first foreign nucleic acid encoding a mutant Cas9 protein including one or more mutations selected from the group consisting of a negatively charged amino acid to a neutral charged amino acid, a negatively charged amino acid to a positively charged amino acid and a neutral charged amino acid to a positively charged amino acid, and
a second foreign nucleic acid encoding a guide RNA and wherein the guide RNA and the mutant Cas9 protein are members of a co-localization complex for a target nucleic acid.

31. The method of claim 30 wherein the one or more mutations are selected from the group consisting of G1104K, L1111H, D1135Y and N1317K.

32. The method of claim 30 wherein the cell is a eukaryotic cell or prokaryotic cell.

33. The method of claim 30 wherein the cell is a bacteria cell, a yeast cell, a fungal cell, a mammalian cell, a plant cell or an animal cell.

34. An RNA guided nucleic acid binding protein having one or more accessory DNA binding domains attached thereto.

35. An RNA guided nucleic acid binding protein having one or more accessory DNA binding domains attached thereto and having target nucleic acid binding activity in the absence of an adjacent functional protospacer adjacent motif.

36. The RNA guided nucleic acid binding protein of claim 35 having nuclease activity.

37. The RNA guided nucleic acid binding protein of claim 35 wherein the RNA guided nucleic acid binding protein is a nickase.

38. The RNA guided nucleic acid binding protein of claim 35 being a nuclease null Cas9 protein.

39. The RNA guided nucleic acid binding protein claim 35 having a transcriptional regulator attached thereto.

40. An RNA guided nucleic acid binding protein having one or more accessory DNA binding domains attached thereto and having increased target nucleic acid binding activity in the presence of an adjacent functional protospacer adjacent motif, compared to wild type RNA guided nucleic acid binding protein.

41. An RNA guided nucleic acid binding protein having one or more accessory DNA binding domains attached thereto bound to a target nucleic acid, wherein the target nucleic acid lacks an adjacent functional protospacer adjacent motif.

42. A method of improving binding of an RNA guided nucleic acid binding protein to a first target nucleic acid comprising
combining an RNA guided nucleic acid binding protein having an accessory DNA binding domain attached thereto, a guide RNA having a spacer sequence complementary to the first target nucleic acid sequence and the first target nucleic acid under conditions where the RNA guided nucleic acid binding protein binds to the first target nucleic acid and the accessory DNA binding domain binds to an accessory target nucleic acid.

43. A method of altering expression of a target nucleic acid in a cell comprising
providing to the cell a Cas9 protein having an accessory DNA binding domain attached thereto,
providing to the cell a guide RNA including a spacer sequence complementary to a target nucleic acid, wherein the guide RNA and the Cas9 protein form a co-localization complex with the target nucleic acid, and
the target nucleic acid is altered.

44. The method of claim 43 wherein the Cas9 protein is an enzymatically active Cas9 and the target nucleic acid is cleaved by the Cas9 protein.

45. The method of claim 43 wherein the Cas9 protein is a nickase and one strand of the target nucleic acid is cleaved by the Cas9 protein.

46. The method of claim 43 wherein the mutant Cas9 protein is a nuclease null Cas9 and wherein a transcriptional regulator is attached to either the Cas9 protein or the guide RNA and the target nucleic acid is regulated.

47. The method of claim 43 wherein the Cas9 protein is provided to the cell by introducing into the cell a first foreign nucleic acid encoding the Cas9 protein and wherein the guide RNA is provided to the cell by introducing into the cell a second foreign nucleic acid encoding the guide RNA, and
wherein the guide RNA and the Cas9 protein are expressed.

48. The method of claim 43 wherein the cell is in vitro, in vivo or ex vivo.

49. The method of claim 43 wherein the cell is a eukaryotic cell or prokaryotic cell.

50. The method of claim 43 wherein the cell is a bacteria cell, a yeast cell, a fungal cell, a mammalian cell, a plant cell or an animal cell.

51. The method of claim 43 wherein the target nucleic acid is genomic DNA, mitochondrial DNA, plastid DNA, viral DNA, or exogenous DNA.

52. A cell comprising
a Cas9 protein having an accessory DNA binding domain attached thereto, and
a guide RNA and wherein the guide RNA and the Cas9 protein are members of a co-localization complex for the target nucleic acid.

53. The method of claim 52 wherein the cell is a eukaryotic cell or prokaryotic cell.

54. The method of claim 52 wherein the cell is a bacteria cell, a yeast cell, a fungal cell, a mammalian cell, a plant cell or an animal cell.

55. A cell comprising
a first foreign nucleic acid encoding a Cas9 protein having an accessory DNA binding domain attached thereto, and
a second foreign nucleic acid encoding a guide RNA and wherein the guide RNA and the Cas9 protein are members of a co-localization complex for a target nucleic acid.

56. The method of claim 55 wherein the cell is a eukaryotic cell or prokaryotic cell.

57. The method of claim 55 wherein the cell is a bacteria cell, a yeast cell, a fungal cell, a mammalian cell, a plant cell or an animal cell.


SEQUENCE LISTING (47 sequences)

SEQ ID NO: 1 - DNA, 30 bp, Artificial Sequence
  Description of Artificial Sequence Synthetic oligonucleotide
  Cas9 cloning plasmid
ttttgctcttctagtggcgggtcagggtcg

SEQ ID NO: 2 - DNA, 15 bp, Artificial Sequence
  Description of Artificial Sequence Synthetic oligonucleotide
  Cas9 cloning plasmid
ttttgctcttctcta

SEQ ID NO: 3 - DNA, 7338 bp, Artificial Sequence
  Description of Artificial Sequence Synthetic polynucleotide
  dCas9-m4-golden gate compatible vector
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgccaccatggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgataaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgagtggtggaggaagttgaagagctatgtttagatatccaaaccaggctcttcttagaagaattcgatccctaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctccatagtcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 4 - DNA, 7338 bp, Artificial Sequence
  Description of Artificial Sequence Synthetic polynucleotide
  Cas9 golden gate compatible vector
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttgccaccatggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgagtggtggaggaagttgaagagctatgtttagatatccaaaccaggctcttcttagaagaattcgatccctaccggttagtaatgagtttaaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctccatagtcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 5 - DNA, 9121 bp, Artificial Sequence
  Description of Artificial Sequence Synthetic polynucleotide
  gRNA cloning vector pSB700-SAM
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcattagtgaacggatctcgacggtatcgatcacgagactagcctcgagcggccgcccccttcaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagacgattaatgcgtctcggttttagagctaggccaacatgaggatcacccatgtctgcagggcctagcaagttaaaataaggctagtccgttatcaacttggccaacatgaggatcacccatgtctgcagggccaagtggcaccgagtcggtgctttttttgaattctcgacctcgagacaaatggcagtattcgtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgttttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 6 - DNA, 20 bp, Artificial Sequence
  Description of Artificial Sequence Synthetic oligonucleotide
  Spacer sequence
ccttggtgaagtctcctttg

SEQ ID NO: 7 - DNA, 20 bp, Artificial Sequence
  Description of Artificial Sequence Synthetic oligonucleotide
  Spacer sequence
atgcgggaggctgagcgcac

SEQ ID NO: 8 - protein, 19 aa, Artificial Sequence
  Description of Artificial Sequence Synthetic peptide
  Ctb peptide sequence
GGSGSSSTSTTAKRKKRKL

SEQ ID NO: 9 - protein, 240 aa, Artificial Sequence
  Description of Artificial Sequence Synthetic polypeptide
  sfGFP+15 peptide sequence
GGASKGERLFTGVVPILVELDGDVNGHKFSVRGEGEGDATRGKLTLKFICTTGKLPVPWPTLVTTLTYGVQCFSRYPKHMKRHDFFKSAMPEGYVQERTISFKKDGTYKTRAEVKFEGRTLVNRIELKGRDFKEKGNILGHKLEYNFNSHNVYITADKRKNGIKANFKIRHNVKDGSVQLADHYQQNTPIGRGPVLLPRNHYLSTRSALSKDPKEKRDHMVLLEFVTAAGITHGMDELYK

SEQ ID NO: 10 - protein, 386 aa, Artificial Sequence
  Description of Artificial Sequence Synthetic polypeptide
  sssIM methyltransferase with methyltransferase point mutation
MSKVENKTKKLRVFEAFAGIGAQRKALEKVRKDEYEIVGLAEWYVPAIVMYQAIHNNFHTKLEYKSVSREEMIDYLENKTLSWNSKNPVSNGYWKRKKDDELKIIYNAIKLSEKEGNIFDIRDLYKRTLKNIDLLTYSFPCQDLSQQGIQKGMKRGSGTRSGLLWEIERALDSTEKNDLPKYLLMANVGALLHKKNEEELNQWKQKLESLGYQNSIEVLNAADFGSSQARRRVFMISTLNEFVELPKGDKKPKSIKKVLNKIVSEKDILNNLLKYNLTEFKKTKSNINKASLIGYSKFNSEGYVYDPEFTGPTLTASGANSRIKIKDGSNIRKMNSDETFLYIGFDSQDGKRVNEIEFLTENQKIFVCGNSISVEVLEAIIDKIGG

SEQ ID NO: 11 - protein, 64 aa, Artificial Sequence
  Description of Artificial Sequence Synthetic polypeptide
  Point mutation sso7d
MATVKFKYKGEEKEVDISKIKKVWRVGKMISFTYDEGGGKTGRGAVSEKDAPKELLQMLEKQKK

SEQ ID NO: 12 - DNA, 20 bp, Artificial Sequence
  Description of Artificial Sequence Synthetic oligonucleotide
tagaggggccgacggagatt

SEQ ID NO: 13 - DNA, 19 bp, Artificial Sequence
  Description of Artificial Sequence Synthetic oligonucleotide
agaaaggaagagtggggaa

SEQ ID NO: 14 - DNA, 19 bp, Artificial Sequence
  Description of Artificial Sequence Synthetic oligonucleotide
acacttccactgtagtcag

SEQ ID NO: 15 - DNA, 20 bp, Artificial Sequence
  Description of Artificial Sequence Synthetic oligonucleotide
taactccttgagtggggcgc

SEQ ID NO: 16 - DNA, 20 bp, Artificial Sequence
  Description of Artificial Sequence Synthetic oligonucleotide
aagagagacagtacatgccc

SEQ ID NO: 17 - DNA, 20 bp, Artificial Sequence
  Description of Artificial Sequence Synthetic oligonucleotide
acgtggatgaaggcgccgcg

SEQ ID NO: 18 - DNA, 20 bp, Artificial Sequence
  Description of Artificial Sequence Synthetic oligonucleotide
ccaggaaaaggactttcaca

SEQ ID NO: 19 - DNA, 21 bp, Artificial Sequence
  Description of Artificial Sequence Synthetic oligonucleotide
gcgccgcagtaaagagagagg

SEQ ID NO: 20 - DNA, 19 bp, Artificial Sequence
  Description of Artificial Sequence Synthetic oligonucleotide
gtctaaccctaactgagaa

SEQ ID NO: 21 - DNA, 21 bp, Artificial Sequence
  Description of Artificial Sequence Synthetic oligonucleotide
gcgcttgtgcggagccggagg

SEQ ID NO: 22 - DNA, 54 bp, Artificial Sequence
  Description of Artificial Sequence Synthetic primer
  modified_base (29)..(34): a, c, t, g, unknown or other
ctttccctacacgacgctcttccgatctnnnnnngcggggactagaccagaagg

SEQ ID NO: 23 - DNA, 54 bp, Artificial Sequence
  Description of Artificial Sequence Synthetic primer
  modified_base (29)..(34): a, c, t, g, unknown or other
ctttccctacacgacgctcttccgatctnnnnnntcaggtttggggctcttttg

SEQ ID NO: 24 - DNA, 54 bp, Artificial Sequence
  Description of Artificial Sequence Synthetic primer
  modified_base (29)..(34): a, c, t, g, unknown or other
ctttccctacacgacgctcttccgatctnnnnnngatccggttagggaggttgg

SEQ ID NO: 25 - DNA, 61 bp, Artificial Sequence
  Description of Artificial Sequence Synthetic primer
  modified_base (29)..(34): a, c, t, g, unknown or other
ctttccctacacgacgctcttccgatctnnnnnntctgtatccttaatggtgttctctctc

SEQ ID NO: 26 - DNA, 54 bp, Artificial Sequence
  Description of Artificial Sequence Synthetic primer
  modified_base (29)..(34): a, c, t, g, unknown or other
ctttccctacacgacgctcttccgatctnnnnnngcacccagaatgaggtggtc

SEQ ID NO: 27 - DNA, 54 bp, Artificial Sequence
  Description of Artificial Sequence Synthetic primer
  modified_base (29)..(34): a, c, t, g, unknown or other
ctttccctacacgacgctcttccgatctnnnnnntccacactctgaggcggaac

SEQ ID NO: 28 - DNA, 54 bp, Artificial Sequence
  Description of Artificial Sequence Synthetic primer
  modified_base (29)..(34): a, c, t, g, unknown or other
ctttccctacacgacgctcttccgatctnnnnnnggagctccagatggctaagg

SEQ ID NO: 29 - DNA, 54 bp, Artificial Sequence
  Description of Artificial Sequence Synthetic primer
  modified_base (29)..(34): a, c, t, g, unknown or other
ctttccctacacgacgctcttccgatctnnnnnnatctttgagggcctgggttg

SEQ ID NO: 30 - DNA, 54 bp, Artificial Sequence
  Description of Artificial Sequence Synthetic primer
  modified_base (29)..(34): a, c, t, g, unknown or other
ctttccctacacgacgctcttccgatctnnnnnncagtggcccgctacaagttc

SEQ ID NO: 31 - DNA, 54 bp, Artificial Sequence
  Description of Artificial Sequence Synthetic primer
  modified_base (29)..(34): a, c, t, g, unknown or other
ctttccctacacgacgctcttccgatctnnnnnntgttctctggtgggcaggag

SEQ ID NO: 32 - DNA, 52 bp, Artificial Sequence
  Description of Artificial Sequence Synthetic primer
  modified_base (29)..(34): a, c, t, g, unknown or other
ctttccctacacgacgctcttccgatctnnnnnncggctgtggctactcagg

SEQ ID NO: 33 - DNA, 53 bp, Artificial Sequence
  Description of Artificial Sequence Synthetic primer
  modified_base (29)..(34): a, c, t, g, unknown or other
ctttccctacacgacgctcttccgatctnnnnnnagccgcgagagtcagcttg

SEQ ID NO: 34 - DNA, 54 bp, Artificial Sequence
  Description of Artificial Sequence Synthetic primer
  modified_base (29)..(34): a, c, t, g, unknown or other
ctttccctacacgacgctcttccgatctnnnnnnctgtgctgagtcggaagtgg

SEQ ID NO: 35 - DNA, 48 bp, Artificial Sequence
  Description of Artificial Sequence Synthetic primer
ggagttcagacgtgtgctcttccgatctggtctgtggattcggtcctc

SEQ ID NO: 36 - DNA, 48 bp, Artificial Sequence
  Description of Artificial Sequence Synthetic primer
ggagttcagacgtgtgctcttccgatctggaaagcatgatgggagagg

SEQ ID NO: 37 - DNA, 48 bp, Artificial Sequence
  Description of Artificial Sequence Synthetic primer
ggagttcagacgtgtgctcttccgatctttgtcctgacactggcatcc

SEQ ID NO: 38 - DNA, 48 bp, Artificial Sequence
  Description of Artificial Sequence Synthetic primer
ggagttcagacgtgtgctcttccgatcttcctggcattgacattccac

SEQ ID NO: 39 - DNA, 48 bp, Artificial Sequence
  Description of Artificial Sequence Synthetic primer
ggagttcagacgtgtgctcttccgatctcatttcccaaatgcgctctc

SEQ ID NO: 40 - DNA, 48 bp, Artificial Sequence
  Description of Artificial Sequence Synthetic primer
ggagttcagacgtgtgctcttccgatctaggcccctgaaagctgctac

SEQ ID NO: 41 - DNA, 48 bp, Artificial Sequence
  Description of Artificial Sequence Synthetic primer
ggagttcagacgtgtgctcttccgatctaccttccagcaggtctgtcg

SEQ ID NO: 42 - DNA, 48 bp, Artificial Sequence
  Description of Artificial Sequence Synthetic primer
ggagttcagacgtgtgctcttccgatctccatgcacctccgtaccttc

SEQ ID NO: 43 - DNA, 47 bp, Artificial Sequence
  Description of Artificial Sequence Synthetic primer
ggagttcagacgtgtgctcttccgatctggttgcggtcgaggaaaag

SEQ ID NO: 44 - DNA, 48 bp, Artificial Sequence
  Description of Artificial Sequence Synthetic primer
ggagttcagacgtgtgctcttccgatcttatgcccagatggcctgaag

SEQ ID NO: 45 - DNA, 49 bp, Artificial Sequence
  Description of Artificial Sequence Synthetic primer
ggagttcagacgtgtgctcttccgatcttctcccctctcacctctagcc

SEQ ID NO: 46 - DNA, 51 bp, Artificial Sequence
  Description of Artificial Sequence Synthetic primer
ggagttcagacgtgtgctcttccgatcttgctctagaatgaacggtggaag

SEQ ID NO: 47 - DNA, 49 bp, Artificial Sequence
  Description of Artificial Sequence Synthetic primer
ggagttcagacgtgtgctcttccgatctccaaatcagacagggtcgaag